(19) United States
(12) Patent Application Publication
(10) Pub. No.: US 2025/0368719 A1
(43) Pub. Date: Dec. 4, 2025

(54) CD6 TARGETED CHIMERIC ANTIGEN RECEPTORS FOR TREATMENT OF CERTAIN AUTOIMMUNE DISORDERS

(71) Applicant: City of Hope, Duarte, CA (US)

(72) Inventors: Jose Enrique Montero Casimiro, Duarte, CA (US); Bart Otto Roep, Duarte, CA (US); Christine E. Brown, Duarte, CA (US)

(21) Appl. No.: 19/178,682

(22) Filed: Apr. 14, 2025

Related U.S. Application Data
(63) Continuation of application No. 17/256,788, filed on Dec. 29, 2020, now Pat. No. 12,281,151.
(60) Provisional application No. 62/692,609, filed on Jun. 29, 2018.

(51) Int. Cl. C07K 14/725; A61K 38/00; A61K 40/11; A61K 40/22; A61K 40/31; A61K 40/41; C07K 14/705; C07K 14/73; C07K 16/28

(52) U.S. Cl. CPC C07K 14/7051; A61K 40/11; A61K 40/22; A61K 40/31; A61K 40/416; C07K 14/70514; C07K 14/70517; C07K 14/70521; C07K 16/2896; A61K 38/00; C07K 2317/53; C07K 2317/56; C07K 2317/565; C07K 2317/622; C07K 2319/02; C07K 2319/03; C07K 2319/30; C07K 2319/33

(57) ABSTRACT

Provided herein are, inter alia, CD6 targeting CAR-T cell compositions and methods useful for treating autoimmune diseases (e.g., Type I diabetes).

### CLAIM OF PRIORITY

This application is a continuation of U.S. application Ser. No. 17/256,788, filed on Dec. 29, 2020, which is a U.S. National Phase Application under 35 U.S.C. §371 of International Application No. PCT/US2019/040185, filed on Jul. 1, 2019, which claims priority to and the benefit of U.S. Provisional Application No. 62/692,609, filed on Jun. 29, 2018. The entire contents of the foregoing are incorporated herein by reference.

### SEQUENCE LISTING

This application contains a Sequence Listing that has been submitted electronically as an XML file named 40056-0051002_SL_ST26.xml. The XML file, created on Apr. 10, 2025, is 100,017 bytes in size. The material in the XML file is hereby incorporated by reference in its entirety.

### BACKGROUND

Type 1 diabetes mellitus (TID) precipitates from the autoimmune attack of pancreatic beta cells thereby resulting in a loss of functional beta cell mass. Functional beta cell mass is impacted positively by processes that increase the number and size of beta cells and negatively by those that deplete the numbers of cells (i.e., apoptosis, necrosis, and other modes of cell death). In addition, beta cell secretory function and capacity is a substantial determinant of functional beta cell mass.

In TID patients, the proliferative and regenerative potential of adult and human islets is low. Thus, it is important to prevent or delay the autoimmune attack and resultant destruction of beta cells, and to establish methods to promote beta cell mass expansion, increase beta cell survival, and/or enhance the function of existing/remaining beta cells, including engaging cellular repair mechanisms to restore functional beta cell mass. However, therapeutic strategies aiming to simultaneously target the pancreatic islet-infiltrating lymphocytes along with protecting and replenishing the functional beta cell mass are limited. Provided herein are solutions to these and other problems in the art.

### SUMMARY

Described herein are chimeric antigen receptors (CARs) targeted to CD6. The CD6 CAR are expressed in regulatory T cells (Tregs) to target the CD6 molecule overexpressed in pro-inflammatory T-cells in Type 1 Diabetes (TID) patients. This approach is in contrast to targeting beta-cells antigens, an approach that may induce additional damage to pancreatic islets. In some instrances the current appoach employs CAR that include an scFv derived from Itolizumab, an immunomodulatory anti-CD6 monoclonal antibody (U.S. Pat. No. 6,572,857). The CD6 CAR used in the CD152 (CTLA-4) cytoplasmic domain (in addition to CD3 zeta) to drive inhibitory signaling in transduced host cells and reinforcing the immunomodulatory activity of CAR-Tregs. In some cases, the CD6 are relatively low affinity with respect to CD6. This can avoid over-activation of adoptively transferred cells and extend their lifespan. In some cases, the CAR are expressed in an CD6low/− subset of Tregs. Thus, in some cases the CD6 CAR are expressed in CD4+, CD25hi,CD127low/−, CD6low/− T cells. In some cases the CD6 CAR expressed in Treg can have a better safety profile compared to CAR expressed in, T effector cells (Teff) because they are less likely to trigger adverse cytokine release syndrome. Moreover, Tregs can produce anti-inflammatory molecules such as IDO, TGF-beta and IL-10. In some cases, the CD6 CAR expressed in Treg are less susceptible to lymphocyte exhaustion resulting in their extended persistence thus improving the efficacy of adoptive immunotherapy.

Provided herein are, inter alia, cells, nucleic acids, proteins, methods, and compositions for an autoimmune disease. In embodiments, the autoimmune disease is associated with reduced islet cell (e.g., beta cell) function, viability, or survival. In embodiments, the autoimmune disease includes a subject's immune system attacking the subject's islet cells (e.g., beta cells). Also provided herein are, inter alia, compositions useful for the treatment of certain autoimmune diseases. In embodiments, novel CAR-T cells targeting the human CD6 molecule are provide herein. In embodiments, chimeric antigen receptors (CARs) with different ranges of affinities for the CD6 molecule are expressed by genetic engineering in different types of human T-cells, including T regulatory cells (Tregs).

In an aspect is provided an isolated nucleic acid encoding a protein including a single chain variable fragment (scFv) targeted to CD6 and a transmembrane domain.

In an aspect, a vector including the nucleic acid provided herein, including embodiments thereof, is provided.

In an aspect, a T lymphocyte, preferably a Treg cell, including the vector provided herein, including embodiments thereof, is provided.

In an aspect, a recombinant protein including a single chain variable fragment (scFv) targeted to CD6 and a transmembrane domain, including embodiments thereof, is provided.

In an aspect, a T lymphocyte, preferably a Treg cell, including the recombinant protein provided herein, including embodiments thereof, is provided.

In an aspect is provided a method of treating an autoimmune disease. In embodiments, the method includes administering to a subject in need thereof an effective amount of a T-lymphocyte, preferably a Treg cell, as described herein, including embodiments thereof.

### DESCRIPTION OF THE DRAWINGS

FIG. 1: Amino acid sequence of the CD6 CAR expressed by pF03496-CD6scFvop(VH_VL)-IgG4(L235E,N297Q)op-CTLA4-Zetaop with the various domains marked. The mature CAR, lacking the signal sequence (MLLLVTSLLLCELPHPAFLLIP; SEQ ID NO:70) is SEQ ID NO:83. The immature sequence is SEQ ID NO:84.

FIG. 2: Amino acid sequence of the CD6 CAR expressed by pF03497-CD6scFvop(VL_VH)-IgG4(L235E,N297Q)op-CTLA4-Zetaop with the various domains marked. The mature CAR, lacking the signal sequence (MLLLVTSLLLCELPHPAFLLIP; SEQ ID NO:70) is SEQ ID NO:85. The immature sequence is SEQ ID NO:86.

FIG. 3: Amino acid sequence of the CD6 CAR expressed by pF03488-CD6scFv(VH-VL)-IgG4op (L235E, N297Q)-41BB-Zetaop with the various domains marked. The mature CAR, lacking the signal sequence (MLLLVTSLLLCELPHPAFLLIP; SEQ ID NO:70) is SEQ ID NO:87. The immature sequence is SEQ ID NO:88.

FIG. 4: Amino acid sequence of the CD6 CAR expressed by pF03491-CD6scFv(VL-VH)-IgG4op(L235E, N297Q)-41BB-Zetaop with the various domains marked. The mature CAR, lacking the signal sequence (MLLLVTSLLLCELPHPAFLLIP; SEQ ID NO:70) is SEQ ID NO:89.The immature sequence is SEQ ID NO:90.

FIG. 5: Is a schematic depiction of the preparation of Tregs. CD25+ cells are isolated from PBMC. FACS sorting is then uses to enrich for CD4⁺/CD25^{high}/CD127^{low/-}. The resulting cells are highly enriched for Tregs. Optionally, an additional step can be used to enrich for the CD6^{low/-} subset of Tregs.

FIG. 6: Is a graph depicting inhibition of the effector T-cells (Teff) proliferation (IC50) by anti-CD6 CAR containing the CD137 (4-1BB) or the CD152 (CTLA-4) cytoplasmic domains, expressed in conventional CD4+CD25hiCD127low/− T regulatory cells (Tregs) host. Circles, conventional Treg-MOCK:Teff-MOCK (IC50=73603); Squares, conventional Treg-CAR (anti-CD6/41BB):Teff-MOCK (IC50=43807): and Diamonds, conventional Treg-CAR (anti-CD6/CTLA4):Teff-MOCK (IC50=14286). Dark, single empty square, Teff-MOCK without stimulus and Light single empty square, Teff-MOCK with stimulus (anti-CD3 OKT3 mAb).

FIG. 7: Depicts the results of an experiment showing the impact of certain CD6 CAR on Teff proliferation.

FIG. 8: Depicts the results of a study examining cytokine expression by Treg expressing certain CD6 CAR.

FIG. 9: Depicts the results of a study showing that Tregs expressing a CD6 CAR with a CTLA4 signaling domain can be cultured in the presence of Teff while expressing relatively low levels of exhaustion markers.

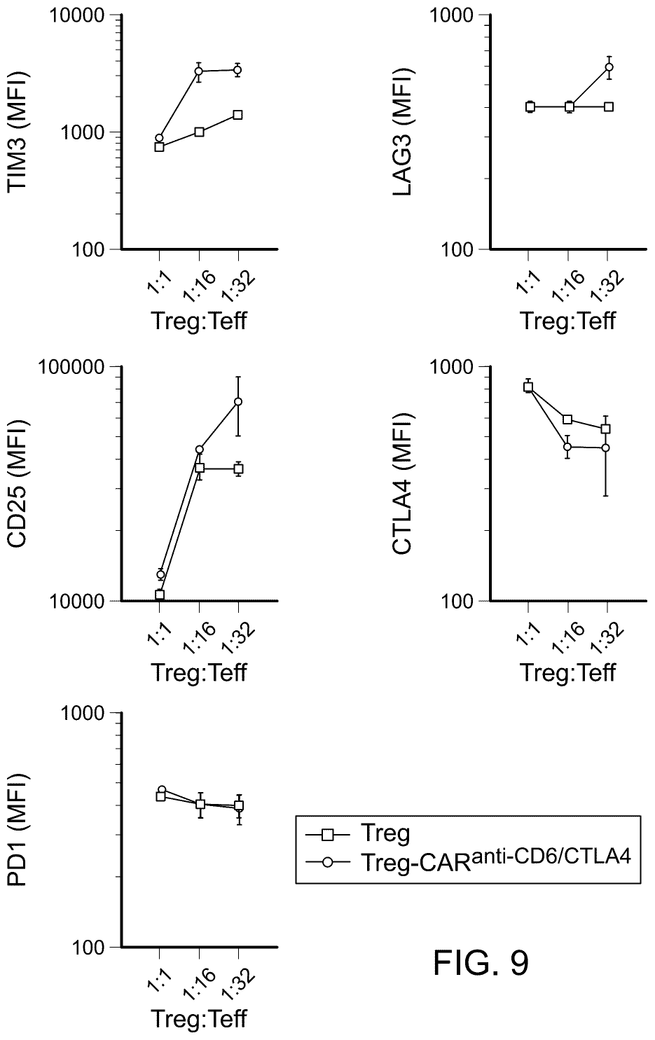

FIG. 10: Amino acid sequence of an alternative CD6 scFv for use in the CAR described herein (SEQ ID NO:73) the domains and mutations are indicated.

### DETAILED DESCRIPTION

Surprisingly, CARs with low affinity slow, delay, or reduce the exhaustion of inoculated CAR-T cells, resulting in a longer half-life and improving efficacy for adoptive immunotherapy. In embodiments, compositions provided herein include CAR-T cells that target CD6+ T- and B-lymphocytes in the affected organs (e.g., pancreas in the case of TID). In embodiments, the CARs provided herein have an affinity, e.g., a KD of 130 nM or higher for the CD6 molecule (i.e., protein). The CAR-T cells provided herein are contemplated as relevant for the treatment of human autoimmune diseases (e.g., Type 1 Diabetes, Multiple Sclerosis, Inflammatory Bowel Disease, or Graft-versus-Host Disease).

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure. The abbreviations used herein have their conventional meaning within the chemical and biological arts. The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The terms “a” or “an,” as used in herein means one or more.

The transitional term “comprising,” which is synonymous with “including,” “containing,” or “characterized by,” is inclusive or open-ended and does not exclude additional, unrecited elements (such as method steps or ingredients). By contrast, the transitional phrase “consisting of” excludes any element not specified in the claim. The transitional phrase “consisting essentially of” limits the scope of a claim to the specified materials or steps “and those that do not materially affect the basic and novel characteristic(s)” of the claimed invention. Where methods and compositions are disclosed using the transitional term “comprising” it will be understood that corresponding methods and compositions with the transitional term “consisting of” and “consisting essentially of” are also disclosed.

Where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

“Nucleic acid” refers to deoxyribonucleotides or ribonucleotides and polymers thereof in either single-or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant.” In embodiments, the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (sec, e.g., Creighton, Proteins (1984)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or antigen binding domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or antigen binding domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using a sequence comparison algorithms or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at about 10 nucleotides in length, or more preferably over a region that is 20 to 50, 100 to 500 or 1000 or more nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. In certain embodiments relating to two sequences of different lengths, the comparison window includes the entire length of the shorter of the two sequences. In some embodiments relating to two sequences of different lengths, the comparison window includes the entire length of the longer of the two sequences.

Methods of alignment of sequences for comparison are well known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Examples of algorithm that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. Software for performing BLAST analyses is publicly available through the National Center for Biotechnology Information (ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In certain embodiments, the NCBI BLASTN or BLASTP program is used to align sequences. In certain embodiments, the BLASTN or BLASTP program uses the defaults used by the NCBI. In certain embodiments, the BLASTN program (for nucleotide sequences) uses as defaults: a word size (W) of 28; an expectation threshold (E) of 10; max matches in a query range set to 0;match/mismatch scores of 1,−2; linear gap costs; the filter for low complexity regions used; and mask for lookup table only used. In certain embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

In certain embodiments, an indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

“Antibody” refers to a polypeptide comprising a framework region from an

immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding site.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a linker peptide (e.g., a short linker peptide of 10 to about 25 amino acids). In embodiments, the linker is rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies or scFvs are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhocyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

A “therapeutic antibody” as provided herein refers to any antibody or functional fragment thereof that is used to treat cancer, autoimmune diseases, transplant rejection, cardiovascular disease or other diseases or conditions such as those described herein.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody. A “therapeutic agent” as referred to herein, is a composition useful in treating or preventing a disease such as an autoimmune disease.

The term “anti-CD6 antibody” as used herein refers to an antibody capable of binding CD6 through the antibody CDR sequences. Thus, the anti-CD6 antibody includes an antibody binding site composed of CDRs CDRs (e.g., VL-CDR1, VL-CDR2, VL-CDR3, VH-CDR1, VH-CDR2, VH-CDR3) that specifically bind CD6

“CD6”, also known as TP120, as referred to herein includes any of the recombinant or naturally-occurring forms of cluster of differentiation 6 (CD6) protein or variants or homologs thereof that maintain CD6 activity (e.g. within at least 50%, 80%, 90%, 95%, 96%, 97%, 98%, 99% or 100% activity compared to CD6). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD6 protein. In some cases, the CD6 protein is substantially identical to the protein identified by the UniProt reference number P30203 or a variant or homolog having substantial identity thereto. In some cases, CD6 is a human CD6 protein. In some cases, a variant or mutant of the CD6 protein includes no more than 5, 4, 3, 2, or 1 deletions compared to a naturally occurring CD6 protein. In some cases, a variant or mutant of the CD6 protein includes no more than 5, 4, 3, 2, or 1 insertions compared to the naturally-occurring CD6 protein. In some cases, a variant or mutant of the CD6 protein does not include deletions compared to the naturally occurring CD6 protein. In some cases, a variant or mutant of the CD6 protein does not include insertions compared to the naturally occurring CD6 protein. In some cases, a variant or mutant of the CD6 protein includes substitutions that are conservative substitutions compared to the naturally occurring CD6 protein. In some cases, CD6 is the protein identified by the NCBI sequence reference NP_006716.3 or an isoform or naturally occurring mutant or variant thereof. In some cases, CD6 is the protein as identified by the NCBI sequence reference NP_001241679.1 or an isoform or naturally occurring mutant or variant thereof. In some cases, CD6 is the protein as identified by the NCBI sequence reference NP_001241680.1 or an isoform or naturally occurring mutant or variant thereof. Non-limiting examples of human CD6 amino acid sequences available under NCBI sequence references are as follows:

NP 006716.3
(SEQ ID NO: 1)
MWLFFGITGLLTAALSGHPSPAPPDQLNTSSAESELWEPGERLPV
RLINGSSSCSGTVEVRLEASWEPACGALWDSRAAEAVCRALGCGG
AEAASQLAPPTPELPPPPAAGNTSVAANATLAGAPALLCSGAEWR
LCEVVEHACRSDGRRARVTCAENRALRLVDGGGACAGRVEMLEHG
EWGSVCDDTWDLEDAHVVCRQLGCGWAVQALPGLHFTPGRGPIHR
DQVNCSGAEAYLWDCPGLPGQHYCGHKEDAGAVCSEHQSWRLTGG
ADRCEGQVEVHFRGVWNTVCDSEWYPSEAKVLCQSLGCGTAVERP
KGLPHSLSGRMYYSCNGEELTLSNCSWRFNNSNLCSQSLAARVLC
SASRSLHNLSTPEVPASVQTVTIESSVTVKIENKESRELMLLIPS
IVLGILLLGSLIFIAFILLRIKGKYALPVMVNHQHLPTTIPAGSN
SYQPVPITIPKEVFMLPIQVQAPPPEDSDSGSDSDYEHYDFSAQP
PVALTTFYNSQRHRVTDEEVQQSRFQMPPLEEGLEELHASHIPTA
NPGHCITDPPSLGPQYHPRSNSESSTSSGEDYCNSPKSKLPPWNP
QVESSERSSFLEQPPNLELAGTQPAFSAGPPADDSSSTSSGEWYQ
NFQPPPQPPSEEQFGCPGSPSPQPDSTDNDDYDDISAA
NP_001241679.1
(SEQ ID NO: 2)
MWLFFGITGLLTAALSGHPSPAPPDQLNTSSAESELWEPGERLPV
RLTNGSSSCSGTVEVRLEASWEPACGALWDSRAAEAVCRALGCGG
AEAASQLAPPTPELPPPPAAGNTSVAANATLAGAPALLCSGAEWR
LCEVVEHACRSDGRRARVTCAENRALRLVDGGGACAGRVEMLEHG
EWGSVCDDTWDLEDAHVVCRQLGCGWAVQALPGLHFTPGRGPIHR
DQVNCSGAEAYLWDCPGLPGQHYCGHKEDAGAVCSEHQSWRLTGG
ADRCEGQVEVHFRGVWNTVCDSEWYPSEAKVLCQSLGCGTAVERP
KGLPHSLSGRMYYSCNGEELTLSNCSWRFNNSNLCSQSLAARVLC
SASRSLHNLSTPEVPASVQTVTIESSVTVKIENKESRELMLLIPS
IVLGILLLGSLIFIAFILLRIKGKYVFMLPIQVQAPPPEDSDSGS
DSDYEHYDFSAQPPVALTTFYNSQRHRVTDEEVQQSRFQMPPLEE
GLEELHASHIPTANPGHCITDPPSLGPQYHPRSNSESSTSSGEDY
CNSPKSKLPPWNPQVFSSERSSFLEQPPNLELAGTQPAFSGSPSP
QPDSTDNDDYDDISAA
NP_001241680.1
(SEQ ID NO: 3)
MWLFFGITGLLTAALSGHPSPAPPDQLNTSSAESELWEPGERLPV
RLINGSSSCSGTVEVRLEASWEPACGALWDSRAAEAVCRALGCGG
AEAASQLAPPTPELPPPPAAGNTSVAANATLAGAPALLCSGAEWR
LCEVVEHACRSDGRRARVTCAENRALRLVDGGGACAGRVEMLEHG
EWGSVCDDTWDLEDAHVVCRQLGCGWAVQALPGLHFTPGRGPIHR
DQVNCSGAEAYLWDCPGLPGQHYCGHKEDAGAVCSEHQSWRLTGG
ADRCEGQVEVHFRGVWNTVCDSEWYPSEAKVLCQSLGCGTAVERP
KGLPHSLSGRMYYSCNGEELTLSNCSWRFNNSNLCSQSLAARVLC
SASRSLHNLSTPEVPASVQTVTIESSVTVKIENKESRELMLLIPS
IVLGILLLGSLIFIAFILLRIKGKYALPVMVNHQHLPTTIPAGSN
SYQPVPITIPKEDSQRHRVTDEEVQQSRFQMPPLEEGLEELHASH
IPTANPGHCITDPPSLGPQYHPRSNSESSTSSGEDYCNSPKSKLP
PWNPQVESSERSSFLEQPPNLELAGTQPAFSGSPSPQPDSTDNDD
YDDISAA

The term “CD28 transmembrane domain” as provided herein includes any of the recombinant or naturally-occurring forms of the transmembrane domain of CD28, or variants or homologs thereof that maintain CD28 transmembrane domain activity (e.g. within at least 50%, 80%, 90%, 95%, 96%, 97%, 98%, 99% or 100% activity compared to the CD28 transmembrane domain). In some cases, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In some cases, the CD28 transmembrane domain is a human CD28 transmembrane domain protein. In some cases, a variant or mutant of the CD28 transmembrane domain protein includes no more than 5, 4, 3, 2, or 1 deletions compared to the naturally occurring CD28 transmembrane domain protein. In some cases, a variant or mutant of the CD28 transmembrane domain protein includes no more than 5, 4, 3, 2, or 1 insertions compared to the naturally occurring CD28 transmembrane domain protein. In some cases, a variant or mutant of the CD28 transmembrane domain protein does not include deletions compared to the naturally occurring CD28 transmembrane domain protein. In some cases, a variant or mutant of the CD28 transmembrane domain protein does not include insertions compared to the naturally occurring CD28 transmembrane domain protein. In some cases, In some cases, a variant or mutant of the CD28 transmembrane domain protein includes substitutions that are conservative substitutions compared to the naturally occurring CD28 transmembrane domain protein. In some cases, In some cases, the CD28 transmembrane domain includes all or a portion of the protein as identified by NCBI sequence reference NP_001230006.1, or an isoform or naturally occurring mutant or variant thereof. In some cases, In some cases, the CD28 transmembrane domain includes all or a portions of the protein as identified by the NCBI sequence reference NP_ 001230007.1, or an isoform or naturally occurring mutant or variant thereof. In some cases, In some cases, the CD28 transmembrane domain includes all or a portion of the protein as identified by the NCBI sequence reference NP_006130.1, or an isoform or naturally occurring mutant or variant thereof. In some cases, In some cases, the CD28 transmembrane domain amino acid sequence includes the sequence of RSKRSRGGHSDYMNMTPRRPGPTRKHYQPYAPPRDFAAYRS (SEQ ID NO:7). In some cases, In some cases, the CD28 transmembrane domain amino acid sequence is the sequence of SEQ ID NO: 7. In some cases, the CD28 transmembrane domain amino acid sequence includes the sequence of RSKRSRLLHSDYMNMTPRRPGPTRKHYQPYAPPRDFAAYRS (SEQ ID NO:8). In some cases, the CD28 transmembrane domain amino acid sequence is the sequence of SEQ ID NO: 8. Non-limiting examples of human CD28 amino acid sequences available under NCBI sequence references are as follows:

NP_001230006.1
(SEQ ID NO: 4)
MLRLLLALNLFPSIQVTGNKILVKQSPMLVAYDNAVNLSWKHLCP
SPLFPGPSKPFWVLVVVGGVLACYSLLVTVAFIIFWVRSKRSRLL
HSDYMNMTPRRPGPTRKHYQPYAPPRDFAAYRS
NP_001230007.1
(SEQ ID NO: 5)
MLRLLLALNLFPSIQVTGKHLCPSPLFPGPSKPFWVLVVVGGVLA
CYSLLVTVAFIIFWVRSKRSRLLHSDYMNMTPRRPGPTRKHYQPY
APPRDFAAYRS
NP_006130.1
(SEQ ID NO: 6)
MLRLLLALNLFPSIQVTGNKILVKQSPMLVAYDNAVNLSCKYSYN
LFSREFRASLHKGLDSAVEVCVVYGNYSQQLQVYSKTGFNCDGKL
GNESVTFYLQNLYVNQTDIYFCKIEVMYPPPYLDNEKSNGTIIHV
KGKHLCPSPLFPGPSKPFWVLVVVGGVLACYSLLVTVAFIIFWVR
SKRSRLLHSDYMNMTPRRPGPTRKHYQPYAPPRDFAAYRS

In some cases, the CD28 transmembrane domain is encoded by all or a portion of the nucleic acid sequence identified by the NCBI sequence reference NM_001243077.1, or an isoform or naturally occurring mutant or variant thereof. In some cases, the CD28 transmembrane domain is encoded by all or a portion the nucleic acid sequence identified by the NCBI sequence reference NM_001243078.1, or an isoform or naturally occurring mutant or variant thereof. In some cases, the CD28 transmembrane domain is encoded by all of a portion of the nucleic acid sequence identified by the NCBI sequence reference NM_006139.3, or an isoform or naturally occurring mutant or variant thereof. In some cases, a variant or mutant of the CD28 transmembrane domain nucleic acid sequence includes no more than 5, 4, 3, 2, or 1 deletions compared to the naturally occurring CD28 transmembrane domain nucleic acid sequence. In some cases, a variant or mutant of the CD28 transmembrane domain nucleic acid sequence includes no more than 5, 4, 3, 2, or 1 insertions compared to the naturally occurring CD28 transmembrane domain nucleic acid sequence. In some cases, a variant or mutant of the CD28 transmembrane domain nucleic acid sequence does not include deletions compared to the naturally occurring CD28 transmembrane domain nucleic acid sequence. In some cases, a variant or mutant of the CD28 transmembrane domain nucleic acid sequence does not include insertions compared to the naturally occurring CD28 transmembrane domain nucleic acid sequence. In some cases, a variant or mutant of the CD28 transmembrane domain nucleic acid sequence includes substitutions that are conservative substitutions compared to the naturally occurring CD28 transmembrane domain nucleic acid sequence.

The term “CD4 transmembrane domain” as provided herein includes any of the recombinant or naturally-occurring forms of the transmembrane domain of CD4, or variants or homologs thereof that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide.In some cases, the CD4 transmembrane domain amino acid sequence includes the sequence of MALIVLGGVAGLLLFIGLGIFF (SEQ ID NO:23). In some cases, the CD4 transmembrane domain amino acid sequence is the sequence of SEQ ID NO:23. A non-limiting example of a nucleotide sequence that encodes the CD4 transmembrane domain is ATGGCCCTGATTGTGCTGGGGGGCGTCGCCGGCCTCCTGCTTTTCATTGGGCTAGGCAT CTTCTTC (SEQ ID NO:24). In some cases, the CD4 transmembrane domain is a human CD4 transmembrane domain protein. In some cases, a variant or mutant of the CD4 transmembrane domain protein includes no more than 5, 4, 3, 2, or 1 deletions compared to the naturally occurring CD4 transmembrane domain protein. In some cases, a variant or mutant of the CD4 transmembrane domain protein includes no more than 5, 4, 3, 2, or 1 insertions compared to the naturally occurring CD4 transmembrane domain protein. In some cases, a variant or mutant of the CD4 transmembrane domain protein does not include deletions compared to the naturally occurring CD4 transmembrane domain protein. In some cases, a variant or mutant of the CD4 transmembrane domain protein does not include insertions compared to the naturally occurring CD4 transmembrane domain protein. In some cases, a variant or mutant of the CD4 transmembrane domain protein includes substitutions that are conservative substitutions compared to the naturally occurring CD4 transmembrane domain protein. In some cases, the CD4 transmembrane domain includes all or a portion of the protein identified by the NCBI sequence reference NP_000607.1, or an isoform or naturally occurring mutant or variant thereof. In some cases, the CD4 transmembrane domain includes all or a portion of the protein identified by the NCBI sequence reference NP_001181943.1, or an isoform or naturally occurring mutant or variant thereof. In some cases, the CD4 transmembrane domain includes all or a portion of the protein identified by the NCBI sequence reference NP_001181944.1, or an isoform or naturally occurring mutant or variant thereof. In some cases, the CD4 transmembrane domain includes all or a portion of the protein identified by the NCBI sequence reference NP_001181945.1, or an isoform or naturally occurring mutant or variant thereof. In some cases, the CD4 transmembrane domain includes all or a portion of the protein identified by the NCBI sequence reference NP_001181946.1, or an isoform or naturally occurring mutant or variant thereof. Non-limiting examples of human CD4 amino acid sequences available under NCBI sequence references are as follows:

NP_000607.1
(SEQ ID NO: 9)
MNRGVPFRHLLLVLQLALLPAATQGKKVVLGKKGDTVELTCTASQ
KKSIQFHWKNSNQIKILGNQGSFLTKGPSKLNDRADSRRSLWDQG
NFPLIIKNLKIEDSDTYICEVEDQKEEVQLLVFGLTANSDTHLLQ
GQSLTLTLESPPGSSPSVQCRSPRGKNIQGGKTLSVSQLELQDSG
TWTCTVLQNQKKVEFKIDIVVLAFQKASSIVYKKEGEQVEFSFPL
AFTVEKLTGSGELWWQAERASSSKSWITFDLKNKEVSVKRVTQDP
KLQMGKKLPLHLTLPQALPQYAGSGNLTLALEAKTGKLHQEVNLV
VMRATQLQKNLTCEVWGPTSPKLMLSLKLENKEAKVSKREKAVWV
LNPEAGMWQCLLSDSGQVLLESNIKVLPTWSTPVQPMALIVLGGV
AGLLLFIGLGIFFCVRCRHRRRQAERMSQIKRLLSEKKTCQCPHR
FQKTCSPI
NP_001181943.1
(SEQ ID NO: 10)
MPTPLVHPHLPISSPRVSPFPPPAFQKASSIVYKKEGEQVEFSFP
LAFTVEKLTGSGELWWQAERASSSKSWITFDLKNKEVSVKRVTQD
PKLQMGKKLPLHLTLPQALPQYAGSGNLTLALEAKTGKLHQEVNL
VVMRATQLQKNLTCEVWGPTSPKLMLSLKLENKEAKVSKREKAVW
VLNPEAGMWQCLLSDSGQVLLESNIKVLPTWSTPVQPMALIVLGG
VAGLLLFIGLGIFFCVRCRHRRRQAERMSQIKRLLSEKKTCQCPH
RFQKTCSPI
NP_001181944.1
(SEQ ID NO: 11)
MGKKLPLHLTLPQALPQYAGSGNLTLALEAKTGKLHQEVNLVVMR
ATQLQKNLTCEVWGPTSPKLMLSLKLENKEAKVSKREKAVWVLNP
EAGMWQCLLSDSGQVLLESNIKVLPTWSTPVQPMALIVLGGVAGL
LLFIGLGIFFCVRCRHRRRQAERMSQIKRLLSEKKTCQCPHRFQK
TCSPI
NP_001181945.1
(SEQ ID NO: 12)
MGKKLPLHLTLPQALPQYAGSGNLTLALEAKTGKLHQEVNLVVMR
ATQLQKNLTCEVWGPTSPKLMLSLKLENKEAKVSKREKAVWVLNP
EAGMWQCLLSDSGQVLLESNIKVLPTWSTPVQPMALIVLGGVAGL
LLFIGLGIFFCVRCRHRRRQAERMSQIKRLLSEKKTCQCPHRFQK
TCSPI
NP_001181946.1
(SEQ ID NO: 13)
MGKKLPLHLTLPQALPQYAGSGNLTLALEAKTGKLHQEVNLVVMR
ATQLQKNLTCEVWGPTSPKLMLSLKLENKEAKVSKREKAVWVLNP
EAGMWQCLLSDSGQVLLESNIKVLPTWSTPVQPMALIVLGGVAGL
LLFIGLGIFFCVRCRHRRRQAERMSQIKRLLSEKKTCQCPHRFQK
TCSPI

In some cases, the CD4 transmembrane domain is encoded by all or a portion of the nucleic acid sequence identified by the NCBI sequence reference NM_000616.4, or an isoform or naturally occurring mutant or variant thereof. In some cases, the CD4 transmembrane domain is encoded by all or a portion of the nucleic acid sequence identified by the NCBI sequence reference NM_001195014.2, or an isoform or naturally occurring mutant or variant thereof. In some cases, the CD4 transmembrane domain is encoded by all or a portion of the nucleic acid sequence identified by the NCBI sequence reference NM_001195015.2, or an isoform or naturally occurring mutant or variant thereof. In some cases, the CD4 transmembrane domain is encoded by all or a portion of the nucleic acid sequence identified by the NCBI sequence reference NM_001195016.2, or an isoform or naturally occurring mutant or variant thereof. In some cases, the CD4 transmembrane domain is encoded by all or a portion of the nucleic acid sequence identified by the NCBI sequence reference NM_001195017.2, or an isoform or naturally occurring mutant or variant thereof. In some cases, a variant or mutant of the CD4 transmembrane domain nucleic acid sequence includes no more than 5, 4, 3, 2, or 1 deletions compared to the naturally occurring CD4 transmembrane domain nucleic acid sequence. In some cases, a variant or mutant of the CD4 transmembrane domain nucleic acid sequence includes no more than 5, 4, 3, 2, or 1 insertions compared to the naturally occurring CD4 transmembrane domain nucleic acid sequence. In some cases, a variant or mutant of the CD4 transmembrane domain nucleic acid sequence does not include deletions compared to the naturally occurring CD4 transmembrane domain nucleic acid sequence. In some cases, a variant or mutant of the CD4 transmembrane domain nucleic acid sequence does not include insertions compared to the naturally occurring CD4 transmembrane domain nucleic acid sequence. In some cases, a variant or mutant of the CD4 transmembrane domain nucleic acid sequence includes substitutions that are conservative substitutions compared to the naturally occurring CD4 transmembrane domain nucleic acid sequence.

The term “CD8 transmembrane domain” as provided herein includes any of the recombinant or naturally-occurring forms of the transmembrane domain of CD8, or variants or homologs thereof that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In some cases, the CD8 transmembrane domain amino acid sequenceincludes the sequence of IYIWAPLAGTCGVLLLSLVIT (SEQ ID NO:25). In some cases, the CD8 transmembrane domain amino acid sequence is the sequence of SEQ ID NO:25. In some cases, the CD8 transmembrane domain is CD8A transmembrane domain. In some cases, the CD8 transmembrane domain is a CD8B transmembrane domain. In some cases, the CD8 transmembrane domain is a human CD8 transmembrane domain protein. In some cases, a variant or mutant of the CD8 transmembrane domain protein includes no more than 5, 4, 3, 2, or 1 deletions compared to the naturally occurring CD8 transmembrane domain protein. In some cases, a variant or mutant of the CD8 transmembrane domain protein includes no more than 5, 4, 3, 2, or 1 insertions compared to the naturally occurring CD8 transmembrane domain protein. In some cases, a variant or mutant of the CD8 transmembrane domain protein does not include deletions compared to the naturally occurring CD8 transmembrane domain protein. In some cases, a variant or mutant of the CD8 transmembrane domain protein does not include insertions compared to the naturally occurring CD8 transmembrane domain protein. In some cases, a variant or mutant of the CD8 transmembrane domain protein includes substitutions that are conservative substitutions compared to the naturally occurring CD8 transmembrane domain protein. In some cases, the CD8 transmembrane domain includes all or a portion of the protein identified by the NCBI sequence reference NP_001139345.1, or an isoform or naturally occurring mutant or variant thereof. In some cases, the CD8 transmembrane domain includes all or a portion of the protein identified by the NCBI sequence reference NP_001181943.1, or an isoform or naturally occurring mutant or variant thereof. In some cases, the CD8 transmembrane domain includes all or a portion of the protein identified by the NCBI sequence reference NP_001181944.1, or an isoform or naturally occurring mutant or variant thereof. In some cases, the CD8 transmembrane domain includes all or a portion of the protein identified by the NCBI sequence reference NP_001181945.1, or an isoform or naturally occurring mutant or variant thereof. In some cases, the CD8 transmembrane domain includes all or a portion of the protein identified by the NCBI sequence reference NP_741969.1, or an isoform or naturally occurring mutant or variant thereof. In some cases, the CD8 transmembrane domain includes all or a portion of the protein identified by the NCBI sequence reference NP_001759.3, or an isoform or naturally occurring mutant or variant thereof. In some cases, the CD8 transmembrane domain includes all or a portion of the protein identified by the NCBI sequence reference XP_011531466.1, or an isoform or naturally occurring mutant or variant thereof. In some cases, the CD8 transmembrane domain includes all or a portion of the protein identified by the NCBI sequence reference NP_001171571.1, or an isoform or naturally occurring mutant or variant thereof. In some cases, the CD8 transmembrane domain includes all or a portion of the protein identified by the NCBI sequence reference NP_757362.1, or an isoform or naturally occurring mutant or variant thereof. In some cases, the CD8 transmembrane domain includes all or a portion of the protein identified by the NCBI sequence reference NP_742100.1, or an isoform or naturally occurring mutant or variant thereof. In some cases, the CD8 transmembrane domain includes all or a portion of the protein identified by the NCBI sequence reference NP_742099.1, or an isoform or naturally occurring mutant or variant thereof. In some cases, the CD8 transmembrane domain includes all or a portion of the protein identified by the NCBI sequence reference NP_004922.1, or an isoform or naturally occurring mutant or variant thereof. Non-limiting examples of human CD8 amino acid sequences available under NCBI sequence references are as follows:

NP_001139345.1
(SEQ ID NO: 46)
MALPVTALLLPLALLLHAARPSQFRVSPLDRTWNLGETVELKCQV
LLSNPTSGCSWLFQPRGAAASPTFLLYLSQNKPKAAEGLDTQRFS
GKRLGDTFVLTLSDFRRENEGYYFCSALSNSIMYFSHFVPVFLPA
KPTTTPAPRPPTPAPTIASQPLSLRPEACRPAAGGAVHTRGLDFA
CDIYIWAPLAGTCGVLLLSLVITLYCNHRNRRRVCKCPRPVVKSG
DKPSLSARYV
NP_741969.1
(SEQ ID NO: 47)
MALPVTALLLPLALLLHAARPSQFRVSPLDRTWNLGETVELKCQV
LLSNPTSGCSWLFQPRGAAASPTFLLYLSQNKPKAAEGLDTQRFS
GKRLGDTFVLTLSDFRRENEGYYFCSALSNSIMYFSHFVPVFLPA
KPTTTPAPRPPTPAPTIASQPLSLRPEACRPAAGGAGNRRRVCKC
PRPVVKSGDKPSLSARYV
NP_001759.3
(SEQ ID NO: 48)
MALPVTALLLPLALLLHAARPSQFRVSPLDRTWNLGETVELKCQV
LLSNPTSGCSWLFQPRGAAASPTFLLYLSQNKPKAAEGLDTQRFS
GKRLGDTFVLTLSDFRRENEGYYFCSALSNSIMYFSHFVPVFLPA
KPTTTPAPRPPTPAPTIASQPLSLRPEACRPAAGGAVHTRGLDFA
CDIYIWAPLAGTCGVLLLSLVITLYCNHRNRRRVCKCPRPVVKSG
DKPSLSARYV
XP_011531466.1
(SEQ ID NO: 49)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGPLCSPITLGLLVAGV
LVLLVSLGVAIHLCCRRRRARLRFMKQKFNIVCLKISGFTTCCCF
QILQMSREYGFGVLLQKDIGQ
NP_001171571.1
(SEQ ID NO: 50)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGLKGKVYQEPLSPNAC
MDTTAILQPHRSCLTHGS
NP_757362.1
(SEQ ID NO: 51)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGPLCSPITLGLLVAGV
LVLLVSLGVAIHLCCRRRRARLRFMKQPQGEGISGTFVPQCLHGY
YSNTTTSQKLLNPWILKT
NP_742100.1
(SEQ ID NO: 52)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGRRRRARLRFMKQPQG
EGISGTFVPQCLHGYYSNTTTSQKLLNPWILKT
NP_742099.1
(SEQ ID NO: 53)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGPLCSPITLGLLVAGV
LVLLVSLGVAIHLCCRRRRARLRFMKQLRLHPLEKCSRMDY
NP_004922.1
(SEQ ID NO: 54)
MRPRLWLLLAAQLTVLHGNSVLQQTPAYIKVQTNKMVMLSCEAKI
SLSNMRIYWLRQRQAPSSDSHHEFLALWDSAKGTIHGEEVEQEKI
AVFRDASRFILNLTSVKPEDSGIYFCMIVGSPELTFGKGTQLSVV
DFLPTTAQPTKKSTLKKRVCRLPRPETQKGPLCSPITLGLLVAGV
LVLLVSLGVAIHLCCRRRRARLRFMKQFYK

In some cases, the CD8 transmembrane domain is encoded by all or a portion of the nucleic acid sequence identified by the NCBI sequence reference NR_027353.1, or an isoform or naturally occurring mutant or variant thereof. In some cases, the CD8 transmembrane domain is encoded by all or a portion of the nucleic acid sequence identified by the NCBI sequence reference NM_001145873.1, or an isoform or naturally occurring mutant or variant thereof. In some cases, the CD8 transmembrane domain is encoded by all or a portion of the nucleic acid sequence identified by the NCBI sequence reference NM_001768.6, or an isoform or naturally occurring mutant or variant thereof. In some cases, the CD8 transmembrane domain is encoded by all or a portion of the nucleic acid sequence identified by the NCBI sequence reference NM_171827.3, or an isoform or naturally occurring mutant or variant thereof. In some cases, the CD8 transmembrane domain is encoded by all or a portion of the nucleic acid sequence identified by the NCBI sequence reference XM_011533164.2, or an isoform or naturally occurring mutant or variant thereof. In some cases, the CD8 transmembrane domain is encoded by all or a portion of the nucleic acid sequence identified by the NCBI sequence reference NM_001178100.1, or an isoform or naturally occurring mutant or variant thereof. In some cases, the CD8 transmembrane domain is encoded by all or a portion of the nucleic acid sequence identified by the NCBI sequence reference NM_004931.4, or an isoform or naturally occurring mutant or variant thereof. In some cases, the CD8 transmembrane domain is encoded by all or a portion of the nucleic acid sequence identified by the NCBI sequence reference NM_172102.3, or an isoform or naturally occurring mutant or variant thereof. In some cases, the CD8 transmembrane domain is encoded by all or a portion of the nucleic acid sequence identified by the NCBI sequence reference NM_172101.3, or an isoform or naturally occurring mutant or variant thereof. In some cases, the CD8 transmembrane domain is encoded by all or a portion of the nucleic acid sequence identified by the NCBI sequence reference NM_172213.3, or an isoform or naturally occurring mutant or variant thereof. In some cases, a variant or mutant of the CD8 transmembrane domain nucleic acid sequence includes no more than 5, 4, 3, 2, or 1 deletions compared to the naturally occurring CD8 transmembrane domain nucleic acid sequence. In some cases, a variant or mutant of the CD8 transmembrane domain nucleic acid sequence includes no more than 5, 4, 3, 2, or 1 insertions compared to the naturally occurring CD8 transmembrane domain nucleic acid sequence. In some cases, a variant or mutant of the CD8 transmembrane domain nucleic acid sequence does not include deletions compared to the naturally occurring CD8 transmembrane domain nucleic acid sequence. In some cases, a variant or mutant of the CD8 transmembrane domain nucleic acid sequence does not include insertions compared to the naturally occurring CD8 transmembrane domain nucleic acid sequence. In some cases, a variant or mutant of the CD8 transmembrane domain nucleic acid sequence includes substitutions that are conservative substitutions compared to the naturally occurring CD8 transmembrane domain nucleic acid sequence.

The term “CD3-zeta transmembrane domain” as provided herein includes any of the recombinant or naturally-occurring forms of the transmembrane domain of CD3-zeta, or variants or homologs thereof that maintain CD3-zeta transmembrane domain activity (e.g. within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In some cases, the CD3-zeta transmembrane domain is a human CD3-zeta transmembrane domain protein. In some cases, a variant or mutant of the CD3-zeta transmembrane domain protein includes no more than 5, 4, 3, 2, or 1 deletions compared to the naturally occurring CD3-zeta transmembrane domain protein. In some cases, a variant or mutant of the CD3-zeta transmembrane domain protein includes no more than 5, 4, 3, 2, or 1 insertions compared to the naturally occurring CD3-zeta transmembrane domain protein. In some cases, a variant or mutant of the CD3-zeta transmembrane domain protein does not include deletions compared to the naturally occurring CD3-zeta transmembrane domain protein. In some cases, a variant or mutant of the CD3-zeta transmembrane domain protein does not include insertions compared to the naturally occurring CD3-zeta transmembrane domain protein. In some cases, a variant or mutant of the CD3-zeta transmembrane domain protein includes substitutions that are conservative substitutions compared to the naturally occurring CD3-zeta transmembrane domain protein. In some cases, the CD3-zeta transmembrane domain includes all or a portion of the protein identified by the NCBI sequence reference NP_000725.1, or an isoform or naturally occurring mutant or variant thereof. In some cases, the CD3-zeta transmembrane domain includes all or a portion of the protein identified by the NCBI sequence reference NP_932170.1, or an isoform or naturally occurring mutant or variant thereof. Non-limiting examples of human CD3-zeta amino acid sequences available under NCBI sequence references are as follows:

NP_000725.1
(SEQ ID NO: 55)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPRRKNPQEGLYNELQKDKMAEAYSEIGMKGERRRGK
GHDGLYQGLSTATKDTYDALHMQALPPR
NP_932170.1
(SEQ ID NO: 56)
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPR

In some cases, the CD3-zeta transmembrane domain is encoded by all or a portion of the nucleic acid sequence identified by the NCBI sequence reference NM_000734.3, or an isoform or naturally occurring mutant or variant thereof. In some cases, the CD3-zeta transmembrane domain is encoded by all or a portion of the nucleic acid sequence identified by the NCBI sequence reference NM_198053.2, or an isoform or naturally occurring mutant or variant thereof. In some cases, a variant or mutant of the CD3-zeta transmembrane domain nucleic acid sequence includes no more than 5, 4, 3, 2, or 1 deletions compared to the naturally occurring CD3-zeta transmembrane domain nucleic acid sequence. In some cases, a variant or mutant of the CD3-zeta transmembrane domain nucleic acid sequence includes no more than 5, 4, 3, 2, or 1 insertions compared to the naturally occurring CD3-zeta transmembrane domain nucleic acid sequence. In some cases, a variant or mutant of the CD3-zeta transmembrane domain nucleic acid sequence does not include deletions compared to the naturally occurring CD3-zeta transmembrane domain nucleic acid sequence. In some cases, a variant or mutant of the CD3-zeta transmembrane domain nucleic acid sequence does not include insertions compared to the naturally occurring CD3-zeta transmembrane domain nucleic acid sequence. In some cases, a variant or mutant of the CD3-zeta transmembrane domain nucleic acid sequence includes substitutions that are conservative substitutions compared to the naturally occurring CD3-zeta transmembrane domain nucleic acid sequence.

The term “CD28 co-stimulatory domain” as provided herein includes any of the recombinant or naturally-occurring forms of the co-stimulatory domain of CD28, or variants or homologs thereof that maintain CD28 co-stimulatory domain activity (e.g. within at least 50%, 80%, 90%, 95%, 96%, 97%, 98%, 99% or 100% activity compared to the CD28 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 co-stimulatory domain polypeptide. In some cases, the CD28 co-stimulatory domain amino acid sequence includes the sequence of RSKRSRGGHSDYMNMTPRRPGPTRKHYQPYAPPRDFAAYRS (SEQID NO: 7). In some cases, the CD28 co-stimulatory domain amino acid sequence is the sequence of RSKRSRLLHSDYMNMTPRRPGPTRKHYQPYAPPRDFAA YRS (SEQ ID NO:8).

In some cases, the CD28 co-stimulatory domain is a human CD28 co-stimulatory domain protein. In some cases, a variant or mutant of the CD28 co-stimulatory domain protein includes no more than 5, 4, 3, 2, or 1 deletions compared to the naturally occurring CD28 co-stimulatory domain protein. In some cases, a variant or mutant of the CD28 co-stimulatory domain protein includes no more than 5, 4, 3, 2, or 1 insertions compared to the naturally occurring CD28 co-stimulatory domain protein. In some cases, a variant or mutant of the CD28 co-stimulatory domain protein does not include deletions compared to the naturally occurring CD28 co-stimulatory domain protein. In some cases, a variant or mutant of the CD28 co-stimulatory domain protein does not include insertions compared to the naturally occurring CD28 co-stimulatory domain protein. In some cases, a variant or mutant of the CD28 co-stimulatory domain protein includes substitutions that are conservative substitutions compared to the naturally occurring CD28 co-stimulatory domain protein. In some cases, the CD28 co-stimulatory domain includes all or a portion of the protein identified by the NCBI sequence reference NP_001230006.1, or an isoform or naturally occurring mutant or variant thereof. In some cases, the CD28 co-stimulatory domain includes all or a portion of the protein identified by the NCBI sequence reference NP_001230007.1, or an isoform or naturally occurring mutant or variant thereof. In some cases, the CD28 co-stimulatory domain includes all or a portion of the protein identified by the NCBI sequence reference NP_006130.1, or an isoform or naturally occurring mutant or variant thereof. Non-limiting examples of human CD28 amino acid sequences available under NCBI sequence references are as follows:

NP_001230006.1
(SEQ ID NO: 57)
MLRLLLALNLFPSIQVTGNKILVKQSPMLVAYDNAVNLSWKHLCP
SPLFPGPSKPFWVLVVVGGVLACYSLLVTVAFIIFWVRSKRSRLL
HSDYMNMTPRRPGPTRKHYQPYAPPRDFAAYRS
NP_001230007.1
(SEQ ID NO: 58)
MLRLLLALNLFPSIQVTGKHLCPSPLFPGPSKPFWVLVVVGGVLA
CYSLLVTVAFIIFWVRSKRSRLLHSDYMNMTPRRPGPTRKHYQPY
APPRDFAAYRS
NP_006130.1
(SEQ ID NO: 59)
MLRLLLALNLFPSIQVTGNKILVKQSPMLVAYDNAVNLSCKYSYN
LFSREFRASLHKGLDSAVEVCVVYGNYSQQLQVYSKTGFNCDGKL
GNESVTFYLQNLYVNQTDIYFCKIEVMYPPPYLDNEKSNGTIIHV
KGKHLCPSPLFPGPSKPFWVLVVVGGVLACYSLLVTVAFIIFWVR
SKRSRLLHSDYMNMTPRRPGPTRKHYQPYAPPRDFAAYRS

In some cases, the CD28 co-stimulatory domain is encoded by all or a portion of the nucleic acid sequence identified by the NCBI sequence reference NM_001243077.1, or an isoform or naturally occurring mutant or variant thereof. In some cases, the CD28 co-stimulatory domain is encoded by all or a portion of the nucleic acid sequence identified by the NCBI sequence reference NM_001243078.1, or an isoform or naturally occurring mutant or variant thereof. In some cases, the CD28 co-stimulatory domain is encoded by all or a portion of the nucleic acid sequence identified by the NCBI sequence reference NM_006139.3, or an isoform or naturally occurring mutant or variant thereof. In some cases, a variant or mutant of the CD28 co-stimulatory domain nucleic acid sequence includes no more than 5, 4, 3, 2, or 1 deletions compared to the naturally occurring CD28 co-stimulatory domain nucleic acid sequence. In some cases, a variant or mutant of the CD28 co-stimulatory domain nucleic acid sequence includes no more than 5, 4, 3, 2, or 1 insertions compared to the naturally occurring CD28 transmembrane domain nucleic acid sequence. In some cases, a variant or mutant of the CD28 co-stimulatory domain nucleic acid sequence does not include deletions compared to the naturally occurring CD28 co-stimulatory domain nucleic acid sequence. In some cases, a variant or mutant of the CD28 co-stimulatory domain nucleic acid sequence does not include insertions compared to the naturally occurring CD28 co-stimulatory domain nucleic acid sequence. In some cases, a variant or mutant of the CD28 co-stimulatory domain nucleic acid sequence includes substitutions that are conservative substitutions compared to the naturally occurring CD28 co-stimulatory domain nucleic acid sequence.

The term “4-1BB co-stimulatory domain” as provided herein includes any of the recombinant or naturally-occurring forms of the co-stimulatory domain of 4-1BB, or variants or homologs thereof that maintain 4-1BB co-stimulatory domain activity (e.g. within at least 50%, 80%, 90%, 95%, 96%, 97%, 98%, 99% or 100% activity compared to the 4-1BB co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring 4-1BB co-stimulatory domain polypeptide. In some cases, the 4-1BB co-stimulatory domain is a human 4-1BB co-stimulatory domain protein. In some cases, a variant or mutant of the 4-1BB co-stimulatory domain protein includes no more than 5, 4, 3, 2, or 1 deletions compared to the naturally occurring 4-1BB co-stimulatory domain protein. In some cases, a variant or mutant of the 4-1BB co-stimulatory domain protein includes no more than 5, 4, 3, 2, or 1 insertions compared to the naturally occurring 4-1BB co-stimulatory domain protein. In some cases, a variant or mutant of the 4-1BB co-stimulatory domain protein does not include deletions compared to the naturally occurring 4-1BB co-stimulatory domain protein. In some cases, a variant or mutant of the 4-1BB co-stimulatory domain protein does not include insertions compared to the naturally occurring 4-1BB co-stimulatory domain protein. In some cases, a variant or mutant of the 4-1BB co-stimulatory domain protein includes substitutions that are conservative substitutions compared to the naturally occurring 4-1BB co-stimulatory domain protein. In some cases, the 4-1BB co-stimulatory domain protein includes the amino acid sequence KRGRKKLLYIFKQPFMRPVQTTQEEDGCSCRFPEEEEGGCEL (SEQ ID NO:14). In some cases, the 4-1BB co-stimulatory domain protein amino acid sequence has at least or about 50%, 60%, 70%, 75%, 80%, 85%, 90%, 91%, 92%, 93%, 94%, 95%, 96%, 97%, 98%, or 99% sequence identify to SEQ ID NO: 14. In some cases, the 4-1BB co-stimulatory domain includes all or a portion of the protein identified by the NCBI sequence reference NP_001552.2 or an isoform or naturally occurring mutant or variant thereof. Non-limiting examples of human 4-1BB amino acid sequences available under NCBI sequence references are as follows:

NP_001552.2
(SEQ ID NO: 15)
MGNSCYNIVATLLLVLNFERTRSLQDPCSNCPAGTFCDNNRNQIC
SPCPPNSESSAGGQRTCDICRQCKGVFRTRKECSSTSNAECDCTP
GFHCLGAGCSMCEQDCKQGQELTKKGCKDCCFGTFNDQKRGICRP
WTNCSLDGKSVLVNGTKERDVVCGPSPADLSPGASSVTPPAPARE
PGHSPQIISFFLALTSTALLFLLFFLTLRFSVVKRGRKKLLYIFK
QPFMRPVQTTQEEDGCSCRFPEEEEGGCEL

In some cases, the 4-1BB co-stimulatory domain is encoded by all or a portion of the nucleic acid sequence identified by the NCBI sequence reference NM_001561.5 or an isoform or naturally occurring mutant or variant thereof. In some cases, a variant or mutant of the 4-1BB co-stimulatory domain nucleic acid sequence includes no more than 5, 4, 3, 2, or 1 deletions compared to the naturally occurring 4-1BB co-stimulatory domain nucleic acid sequence. In some cases, a variant or mutant of the 4-1BB co-stimulatory domain nucleic acid sequence includes no more than 5, 4, 3, 2, or 1 insertions compared to the naturally occurring 4-1BB co-stimulatory domain nucleic acid sequence. In some cases, a variant or mutant of the 4-1BB co-stimulatory domain nucleic acid sequence does not include deletions compared to the naturally occurring 4-1BB co-stimulatory domain nucleic acid sequence. In some cases, a variant or mutant of the 4-1BB co-stimulatory domain nucleic acid sequence does not include insertions compared to the naturally occurring 4-1BB co-stimulatory domain nucleic acid sequence. In some cases, a variant or mutant of the 4-1BB co-stimulatory domain nucleic acid sequence includes substitutions that are conservative substitutions compared to the naturally occurring 4-1BB co-stimulatory domain nucleic acid sequence.

The term “ICOS co-stimulatory domain” as provided herein includes any of the recombinant or naturally-occurring forms of the co-stimulatory domain of ICOS, or variants or homologs thereof that maintain ICOS co-stimulatory domain activity (e.g. within at least 50%, 80%, 90%, 95%, 96%, 97%, 98%, 99% or 100% activity compared to the ICOS co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ICOS co-stimulatory domain polypeptide. In some cases, the ICOS co-stimulatory domain amino acid sequence includes the sequence of CWLTKKKYSSSVHDPNGEYMFMRAVNTAKKSRLTDVTL (SEQ ID NO:31). In some cases, the ICOS co-stimulatory domain is a human ICOS co-stimulatory domain protein. In some cases, a variant or mutant of the ICOS co-stimulatory domain protein includes no more than 5, 4, 3, 2, or 1 deletions compared to the naturally occurring ICOS co-stimulatory domain protein. In some cases, a variant or mutant of the ICOS co-stimulatory domain protein includes no more than 5, 4, 3, 2, or 1 insertions compared to the naturally occurring ICOS co-stimulatory domain protein. In some cases, a variant or mutant of the ICOS co-stimulatory domain protein does not include deletions compared to the naturally occurring ICOS co-stimulatory domain protein. In some cases, a variant or mutant of the ICOS co-stimulatory domain protein does not include insertions compared to the naturally occurring ICOS co-stimulatory domain protein. In some cases, a variant or mutant of the ICOS co-stimulatory domain protein includes substitutions that are conservative substitutions compared to the naturally occurring ICOS co-stimulatory domain protein. In some cases, the ICOS co-stimulatory domain includes all or a portion of the protein identified by the NCBI sequence reference NP_036224.1, or an isoform or naturally occurring mutant or variant thereof. Non-limiting examples of human ICOS amino acid sequences available under NCBI sequence references are as follows:

NP_036224.1
(SEQ ID NO: 16)
MKSGLWYFFLFCLRIKVLTGEINGSANYEMFIFHNGGVQILCKYP
DIVQQFKMQLLKGGQILCDLTKTKGSGNTVSIKSLKFCHSQLSNN
SVSFFLYNLDHSHANYYFCNLSIFDPPPFKVTLTGGYLHIYESQL
CCQLKFWLPIGCAAFVVVCILGCILICWLTKKKYSSSVHDPNGEY
MFMRAVNTAKKSRLTDVTL

In some cases, the ICOS co-stimulatory domain is encoded by all or a portion of the nucleic acid sequence identified by the NCBI sequence reference NM_012092.3, or an isoform or naturally occurring mutant or variant thereof. In some cases, a variant or mutant of the ICOS co-stimulatory domain nucleic acid sequence includes no more than 5, 4, 3, 2, or 1 deletions compared to the naturally occurring ICOS co-stimulatory domain nucleic acid sequence. In some cases, a variant or mutant of the ICOS co-stimulatory domain nucleic acid sequence includes no more than 5, 4, 3, 2, or 1 insertions compared to the naturally occurring ICOS co-stimulatory domain nucleic acid sequence. In some cases, a variant or mutant of the ICOS co-stimulatory domain nucleic acid sequence does not include deletions compared to the naturally occurring ICOS co-stimulatory domain nucleic acid sequence. In some cases, a variant or mutant of the ICOS co-stimulatory domain nucleic acid sequence does not include insertions compared to the naturally occurring ICOS co-stimulatory domain nucleic acid sequence. In some cases, a variant or mutant of the ICOS co-stimulatory domain nucleic acid sequence includes substitutions that are conservative substitutions compared to the naturally occurring ICOS co-stimulatory domain nucleic acid sequence.

The term “OX-40 co-stimulatory domain” as provided herein includes any of the recombinant or naturally-occurring forms of the co-stimulatory domain of OX-40, or variants or homologs thereof that maintain OX-40 co-stimulatory domain activity (e.g. within at least 50%, 80%, 90%, 95%, 96%, 97%, 98%, 99% or 100% activity compared to the OX-40 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring OX-40 co-stimulatory domain polypeptide. In some cases, the OX-40 co-stimulatory domain amino acid sequence includes the sequence of ALYLLRRDQRLPPDAHKPPGGGSFRTPIQEEQADAHSTLAKI (SEQ ID NO:32). In some cases, the OX-40 co-stimulatory domain is a human OX-40 co-stimulatory domain protein. In some cases, a variant or mutant of the OX-40 co-stimulatory domain protein includes no more than 5, 4, 3, 2, or 1 deletions compared to the naturally occurring OX-40 co-stimulatory domain protein. In some cases, a variant or mutant of the OX-40 co-stimulatory domain protein includes no more than 5, 4, 3, 2, or 1 insertions compared to the naturally occurring OX-40 co-stimulatory domain protein. In some cases, a variant or mutant of the OX-40 co-stimulatory domain protein does not include deletions compared to the naturally occurring OX-40 co-stimulatory domain protein. In some cases, a variant or mutant of the OX-40 co-stimulatory domain protein does not include insertions compared to the naturally occurring OX-40 co-stimulatory domain protein. In some cases, a variant or mutant of the OX-40 co-stimulatory domain protein includes substitutions that are conservative substitutions compared to the naturally occurring OX-40 co-stimulatory domain protein. In some cases, the OX-40 co-stimulatory domain includes all or a portion of the protein identified by the NCBI sequence reference NP_003318.1, or an isoform or naturally occurring mutant or variant thereof. Non-limiting examples of human OX-40 amino acid sequences available under NCBI sequence references are as follows:

NP_003318.1
(SEQ ID NO: 60)
MCVGARRLGRGPCAALLLLGLGLSTVTGLHCVGDTYPSNDRCCHE
CRPGNGMVSRCSRSQNTVCRPCGPGFYNDVVSSKPCKPCTWCNLR
SGSERKQLCTATQDTVCRCRAGTQPLDSYKPGVDCAPCPPGHFSP
GDNQACKPWTNCTLAGKHTLQPASNSSDAICEDRDPPATQPQETQ
GPPARPITVQPTEAWPRTSQGPSTRPVEVPGGRAVAAILGLGLVL
GLLGPLAILLALYLLRRDQRLPPDAHKPPGGGSFRTPIQEEQADA
HSTLAKI

In some cases, the OX-40 co-stimulatory domain is encoded by all or a portion of the nucleic acid sequence identified by the NCBI sequence reference NM_003327.3, or an isoform or naturally occurring mutant or variant thereof. In some cases, a variant or mutant of the OX-40 co-stimulatory domain nucleic acid sequence includes no more than 5, 4, 3, 2, or 1 deletions compared to the naturally occurring OX-40 co-stimulatory domain nucleic acid sequence. In some cases, a variant or mutant of the OX-40 co-stimulatory domain nucleic acid sequence includes no more than 5, 4, 3, 2, or 1 insertions compared to the naturally occurring OX-40 co-stimulatory domain nucleic acid sequence. In some cases, a variant or mutant of the OX-40 co-stimulatory domain nucleic acid sequence does not include deletions compared to the naturally occurring OX-40 co-stimulatory domain nucleic acid sequence. In some cases, a variant or mutant of the OX-40 co-stimulatory domain nucleic acid sequence does not include insertions compared to the naturally occurring OX-40 co-stimulatory domain nucleic acid sequence. In some cases, a variant or mutant of the OX-40 co-stimulatory domain nucleic acid sequence includes substitutions that are conservative substitutions compared to the naturally occurring OX-40 co-stimulatory domain nucleic acid sequence.

The term “CTLA-4 co-stimulatory domain” as provided herein includes any of the recombinant or naturally-occurring forms of the co-stimulatory domain of CTLA-4, or variants or homologs thereof that maintain CTLA-4 co-stimulatory domain activity (e.g. within at least 50%, 80%, 90%, 95%, 96%, 97%, 98%, 99% or 100% activity compared to the CTLA-4 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 co-stimulatory domain polypeptide. In some cases, CTLA-4 co-stimulatory domain protein is a human CTLA-4 co-stimulatory domain protein. In some cases, the CTLA-4 co-stimulatory domain includes no more than 5, 4, 3, 2, or 1 deletions. In some cases, the CTLA-4 co-stimulatory domain protein includes no more than 5, 4, 3, 2, or 1 insertions. In some cases, the CTLA-4 co-stimulatory domain protein does not include deletions. In some cases, CTLA-4 co-stimulatory domain protein does not include insertions. In some cases, the CTLA-4 co-stimulatory domain protein includes substitutions that are conservative substitutions. In some cases, CTLA-4 co-stimulatory domain protein includes the sequence AVSLSKMLKKRSPLTTGVYVKMPPTEPECEKQFQPYFIPIN (SEQ ID NO:17). In some cases, CTLA-4 co-stimulatory domain protein is the sequence SEQ ID NO: 17. In some cases, the CTLA-4 co-stimulatory domain amino acid sequence has at least or about 50%, 60%, 70%, 75%, 80%, 85%, 90%, 91%, 92%, 93%, 94%, 95%, 96%, 97%, 98%, or 99% sequence identify to SEQ ID NO:17. In some cases, the CTLA-4 co-stimulatory domain is a human CTLA-4 co-stimulatory domain protein. In some cases, a variant or mutant of the CTLA-4 co-stimulatory domain protein includes no more than 5, 4, 3, 2, or 1 deletions compared to the naturally occurring CTLA-4 co-stimulatory domain protein. In some cases, a variant or mutant of the CTLA-4 co-stimulatory domain protein includes no more than 5, 4, 3, 2, or 1 insertions compared to the naturally occurring CTLA-4 co-stimulatory domain protein. In some cases, a variant or mutant of the CTLA-4 co-stimulatory domain protein does not include deletions compared to the naturally occurring CTLA-4 co-stimulatory domain protein. In some cases, a variant or mutant of the CTLA-4 co-stimulatory domain protein does not include insertions compared to the naturally occurring CTLA-4 co-stimulatory domain protein. In some cases, a variant or mutant of the CTLA-4 co-stimulatory domain protein includes substitutions that are conservative substitutions compared to the naturally occurring CTLA-4 co-stimulatory domain protein. In some cases, the CTLA-4 co-stimulatory domain includes all or a portion of the protein identified by the NCBI sequence reference NP_001032720.1, or an isoform or naturally occurring mutant or variant thereof. In some cases, the CTLA-4 co-stimulatory domain includes all or a portion of the protein identified by the NCBI sequence reference NP_005205.2, or an isoform or naturally occurring mutant or variant thereof. Non-limiting examples of human CTLA-4 amino acid sequences available under NCBI sequence references are as follows:

NP_001032720.1
(SEQ ID NO: 61)
MACLGFQRHKAQLNLATRTWPCTLLFFLLFIPVFCKAMHVAQPAV
VLASSRGIASFVCEYASPGKATEVRVTVLRQADSQVTEVCAATYM
MGNELTFLDDSICTGTSSGNQVNLTIQGLRAMDTGLYICKVELMY
PPPYYLGIGNGTQIYVIAKEKKPSYNRGLCENAPNRARM
NP_005205.2
(SEQ ID NO: 62)
MACLGFQRHKAQLNLATRTWPCTLLFFLLFIPVFCKAMHVAQPAV
VLASSRGIASFVCEYASPGKATEVRVTVLRQADSQVTEVCAATYM
MGNELTFLDDSICTGTSSGNQVNLTIQGLRAMDTGLYICKVELMY
PPPYYLGIGNGTQIYVIDPEPCPDSDFLLWILAAVSSGLFFYSFL
LTAVSLSKMLKKRSPLTTGVYVKMPPTEPECEKQFQPYFIPIN

In some cases, the CTLA-4 co-stimulatory domain is encoded by all or a portion of the nucleic acid sequence identified by the NCBI sequence reference NM_001037631.3, or an isoform or naturally occurring mutant or variant thereof. In some cases, the CTLA-4 co-stimulatory domain is encoded by all or a portion of the nucleic acid sequence identified by the NCBI sequence reference NM_005214.5, or an isoform or naturally occurring mutant or variant thereof. In some cases, a variant or mutant of the CTLA-4 co-stimulatory domain nucleic acid sequence includes no more than 5, 4, 3, 2, or 1 deletions compared to the naturally occurring CTLA-4 co-stimulatory domain nucleic acid sequence. In some cases, a variant or mutant of the CTLA-4 co-stimulatory domain nucleic acid sequence includes no more than 5, 4, 3, 2, or 1 insertions compared to the naturally occurring CTLA-4 co-stimulatory domain nucleic acid sequence. In some cases, a variant or mutant of the CTLA-4 co-stimulatory domain nucleic acid sequence does not include deletions compared to the naturally occurring CTLA-4 co-stimulatory domain nucleic acid sequence. In some cases, a variant or mutant of the CTLA-4 co-stimulatory domain nucleic acid sequence does not include insertions compared to the naturally occurring CTLA-4 co-stimulatory domain nucleic acid sequence. In some cases, a variant or mutant of the CTLA-4 co-stimulatory domain nucleic acid sequence includes substitutions that are conservative substitutions compared to the naturally occurring CTLA-4 co-stimulatory domain nucleic acid sequence.

The term “PD-1 co-stimulatory domain” as provided herein includes any of the recombinant or naturally-occurring forms of the co-stimulatory domain of PD-1, or variants or homologs thereof that maintain PD-1 co-stimulatory domain activity (e.g. within at least 50%, 80%, 90%, 95%, 96%, 97%, 98%, 99% or 100% activity compared to the PD-1 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 co-stimulatory domain polypeptide. In some cases, the PD-I co-stimulatory domain amino acid sequence includes the sequence of CSRAARGTIGARRTGQPLKEDPSAVPVFSVDYGELDFQWREKTPEPPVPCVPEQTEYAT IVFPSGMGTSSP ARRGSADGPRSAQPLRPEDGHCSWPL (SEQ ID NO:33). In some cases, the PD-1 co-stimulatory domain” is a human PD-1 co-stimulatory domain protein. In some cases, a variant or mutant of the PD-1 co-stimulatory domain protein includes no more than 5, 4, 3, 2, or 1 deletions compared to the naturally occurring PD-1 co-stimulatory domain protein. In some cases, a variant or mutant of the PD-1 co-stimulatory domain protein includes no more than 5, 4, 3, 2, or 1 insertions compared to the naturally occurring PD-1 co-stimulatory domain protein. In some cases, a variant or mutant of the PD-1 co-stimulatory domain protein does not include deletions compared to the naturally occurring PD-1 co-stimulatory domain protein. In some cases, a variant or mutant of the PD-1 co-stimulatory domain protein does not include insertions compared to the naturally occurring PD-1 co-stimulatory domain protein. In some cases, a variant or mutant of the PD-1 co-stimulatory domain protein includes substitutions that are conservative substitutions compared to the naturally occurring PD-1 co-stimulatory domain protein. In some cases, the PD-1 co-stimulatory domain includes all or a portion of the protein identified by the NCBI sequence reference NP_005009.2, or an isoform or naturally occurring mutant or variant thereof. Non-limiting examples of human PD-1 amino acid sequences available under NCBI sequence references are as follows:

NP_005009.2
(SEQ ID NO: 63)
MQIPQAPWPVVWAVLQLGWRPGWFLDSPDRPWNPPTFSPALLVVT
EGDNATFTCSFSNTSESFVLNWYRMSPSNQTDKLAAFPEDRSQPG
QDCRFRVTQLPNGRDFHMSVVRARRNDSGTYLCGAISLAPKAQIK
ESLRAELRVTERRAEVPTAHPSPSPRPAGQFQTLVVGVVGGLLGS
LVLLVWVLAVICSRAARGTIGARRTGQPLKEDPSAVPVFSVDYGE
LDFQWREKTPEPPVPCVPEQTEYATIVFPSGMGTSSPARRGSADG
PRSAQPLRPEDGHCSWPL

In some cases, the PD-1 co-stimulatory domain is encoded by all or a portion of the nucleic acid sequence identified by the NCBI sequence reference NM_005018.2, or an isoform or naturally occurring mutant or variant thereof. In some cases, a variant or mutant of the PD-1 co-stimulatory domain nucleic acid sequence includes no more than 5, 4, 3, 2, or 1 deletions compared to the naturally occurring PD-1 co-stimulatory domain nucleic acid sequence. In some cases, a variant or mutant of the PD-1 co-stimulatory domain nucleic acid sequence includes no more than 5, 4, 3, 2, or 1 insertions compared to the naturally occurring PD-1 co-stimulatory domain nucleic acid sequence. In some cases, a variant or mutant of the PD-1 co-stimulatory domain nucleic acid sequence does not include deletions compared to the naturally occurring PD-1 co-stimulatory domain nucleic acid sequence. In some cases, a variant or mutant of the PD-1 co-stimulatory domain nucleic acid sequence does not include insertions compared to the naturally occurring PD-1 co-stimulatory domain nucleic acid sequence. In some cases, a variant or mutant of the PD-1 co-stimulatory domain nucleic acid sequence includes substitutions that are conservative substitutions compared to the naturally occurring PD-1 co-stimulatory domain nucleic acid sequence.

The term “GITR co-stimulatory domain” as provided herein includes any of the recombinant or naturally-occurring forms of the co-stimulatory domain of GITR, or variants or homologs thereof that maintain GITR co-stimulatory domain activity (e.g. within at least 50%, 80%, 90%, 95%, 96%, 97%, 98%, 99% or 100% activity compared to the GITR co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring GITR co-stimulatory domain polypeptide. In some cases, the GITR co-stimulatory domain amino acid sequence includes the sequence of QLGLHIWQLRSQCMWPRETQLLLEVPPSTEDARSCQFPEEERGERSAEEKGRLGDLWV (SEQ ID NO:67) In some cases, the GITR co-stimulatory domain is a human GITR co-stimulatory domain protein. In some cases, a variant or mutant of the GITR co-stimulatory domain protein includes no more than 5, 4, 3, 2, or 1 deletions compared to the naturally occurring GITR co-stimulatory domain protein. In some cases, a variant or mutant of the GITR co-stimulatory domain protein includes no more than 5, 4, 3, 2, or 1 insertions compared to the naturally occurring GITR co-stimulatory domain protein. In some cases, a variant or mutant of the GITR co-stimulatory domain protein does not include deletions compared to the naturally occurring GITR co-stimulatory domain protein. In some cases, a variant or mutant of the GITR co-stimulatory domain protein does not include insertions compared to the naturally occurring GITR co-stimulatory domain protein. In some cases, a variant or mutant of the GITR co-stimulatory domain protein includes substitutions that are conservative substitutions compared to the naturally occurring GITR co-stimulatory domain protein. In some cases, the GITR co-stimulatory domain includes all or a portion of the protein identified by the NCBI sequence reference NP_004186.1, or an isoform or naturally occurring mutant or variant thereof. In some cases, the GITR co-stimulatory domain includes all or a portion of the protein identified by the NCBI sequence reference NP_683699.1, or an isoform or naturally occurring mutant or variant thereof. In some cases, the GITR co-stimulatory domain includes all or a portion of the protein identified by the NCBI sequence reference NP_683700.1, or an isoform or naturally occurring mutant or variant thereof. Non-limiting examples of human GITR amino acid sequences available under NCBI sequence references are as follows:

NP_004186.1
(SEQ ID NO: 64)
MAQHGAMGAFRALCGLALLCALSLGQRPTGGPGCGPGRLLLGTGT
DARCCRVHTTRCCRDYPGEECCSEWDCMCVQPEFHCGDPCCTTCR
HHPCPPGQGVQSQGKFSFGFQCIDCASGTFSGGHEGHCKPWTDCT
QFGFLTVFPGNKTHNAVCVPGSPPAEPLGWLTVVLLAVAACVLLL
TSAQLGLHIWQLRSQCMWPRETQLLLEVPPSTEDARSCQFPEEER
GERSAEEKGRLGDLWV
NP_683699.1
(SEQ ID NO: 65)
MAQHGAMGAFRALCGLALLCALSLGQRPTGGPGCGPGRLLLGTGT
DARCCRVHTTRCCRDYPGEECCSEWDCMCVQPEFHCGDPCCTTCR
HHPCPPGQGVQSQGKFSFGFQCIDCASGTFSGGHEGHCKPWTDCC
WRCRRRPKTPEAASSPRKSGASDRQRRRGGWETCGCEPGRPPGPP
TAASPSPGAPQAAGALRSALGRALLPWQQKWVQEGGSDQRPGPCS
SAAAAGPCRRERETQSWPPSSLAGPDGVGS
NP_683700.1
(SEQ ID NO: 66)
MAQHGAMGAFRALCGLALLCALSLGQRPTGGPGCGPGRLLLGTGT
DARCCRVHTTRCCRDYPGEECCSEWDCMCVQPEFHCGDPCCTTCR
HHPCPPGQGVQSQGKFSFGFQCIDCASGTFSGGHEGHCKPWTDCT
QFGFLTVFPGNKTHNAVCVPGSPPAEPLGWLTVVLLAVAACVLLL
TSAQLGLHIWQLRKTQLLLEVPPSTEDARSCQFPEEERGERSAEE
KGRLGDLWV

In some cases, the GITR co-stimulatory domain is encoded by all or a portion of the nucleic acid sequence identified by the NCBI sequence reference NM_148902.1, or an isoform or naturally occurring mutant or variant thereof. In some cases, the GITR co-stimulatory domain is encoded by all or a portion of the nucleic acid sequence identified by the NCBI sequence reference NM_004195.2, or an isoform or naturally occurring mutant or variant thereof. In some cases, the GITR co-stimulatory domain is encoded by all or a portion of the nucleic acid sequence identified by the NCBI sequence reference NM_148901.1, or an isoform or naturally occurring mutant or variant thereof. In some cases, a variant or mutant of the GITR co-stimulatory domain nucleic acid sequence includes no more than 5, 4, 3, 2, or 1 deletions compared to the naturally occurring GITR co-stimulatory domain nucleic acid sequence. In some cases, a variant or mutant of the GITR co-stimulatory domain nucleic acid sequence includes no more than 5, 4, 3, 2, or 1 insertions compared to the naturally occurring GITR co-stimulatory domain nucleic acid sequence. In some cases, a variant or mutant of the GITR co-stimulatory domain nucleic acid sequence does not include deletions compared to the naturally occurring GITR co-stimulatory domain nucleic acid sequence. In some cases, a variant or mutant of the GITR co-stimulatory domain nucleic acid sequence does not include insertions compared to the naturally occurring GITR co-stimulatory domain nucleic acid sequence. In some cases, a variant or mutant of the GITR co-stimulatory domain nucleic acid sequence includes substitutions that are conservative substitutions compared to the naturally occurring GITR co-stimulatory domain nucleic acid sequence.

The term “CD3ζ intracellular T-cell signaling domain” as provided herein includes any of the recombinant or naturally-occurring forms of the CD3ζ intracellular T-cell signaling domain, or variants or homologs thereof that maintain CD3ζ intracellular T-cell signaling domain activity (e.g. within at least 50%, 80%, 90%, 95%, 96%, 97%, 98%, 99% or 100% activity compared to the CD3ζ intracellular T-cell signaling domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3ζ intracellular T-cell signaling domain polypeptide. In some cases, the CD3ζ intracellular T-cell signaling domain is a human CD3ζ intracellular T-cell signaling domain protein. In some cases, a variant or mutant of the CD3ζ intracellular T-cell signaling domain protein includes no more than 5, 4, 3, 2, or 1 deletions compared to the naturally occurring CD3ζ intracellular T-cell signaling domain protein. In some cases, a variant or mutant of the CD3ζ intracellular T-cell signaling domain protein includes no more than 5, 4, 3, 2, or 1 insertions compared to the naturally occurring CD3ζ intracellular T-cell signaling domain protein. In some cases, a variant or mutant of the CD3ζ intracellular T-cell signaling domain protein does not include deletions compared to the naturally occurring CD3 intracellular T-cell signaling domain protein. In some cases, a variant or mutant of the CD3ζ intracellular T-cell signaling domain protein does not include insertions compared to the naturally occurring CD3ζ intracellular T-cell signaling domain protein. In some cases, a variant or mutant of the CD3ζ intracellular T-cell signaling domain protein includes substitutions that are conservative substitutions compared to the naturally occurring CD35intracellular T-cell signaling domain protein. In some cases, the CD3ζ intracellular T-cell signaling domain includes all or a portion of the protein identified by the NCBI sequence reference NP_000725.1, or an isoform or naturally occurring mutant or variant thereof. In some cases, the CD3ζ intracellular T-cell signaling domain includes all or a portion of the protein identified by the NCBI sequence reference NP_932170.1, or an isoform or naturally occurring mutant or variant thereof. Non-limiting examples of human CD3-zeta amino acid sequences available under NCBI sequence references are identified supra. In some cases, the CD3 intracellular T-cell signaling domain is encoded by all or a portion of the nucleic acid sequence identified by the NCBI sequence reference NM_000734.3, or an isoform or naturally occurring mutant or variant thereof. In some cases, the CD3 intracellular T-cell signaling domain is encoded by all or a portion of the nucleic acid sequence identified by the NCBI sequence reference NM_198053.2, or an isoform or naturally occurring mutant or variant thereof. In some cases, a variant or mutant of the CD3ζ intracellular T-cell signaling domain nucleic acid sequence includes no more than 5, 4, 3, 2, or 1 deletions compared to the naturally occurring CD3 (intracellular T-cell signaling domain nucleic acid sequence. In some cases, a variant or mutant of the CD3ζ intracellular T-cell signaling domain nucleic acid sequence includes no more than 5, 4, 3, 2, or 1 insertions compared to the naturally occurring CD3ζ intracellular T-cell signaling domain nucleic acid sequence. In some cases, a variant or mutant of the CD3ζ intracellular T-cell signaling domain nucleic acid sequence does not include deletions compared to the naturally occurring CD3ζ intracellular T-cell signaling domain nucleic acid sequence. In some cases, a variant or mutant of the CD3ζ intracellular T-cell signaling domain nucleic acid sequence does not include insertions compared to the naturally occurring CD3ζ intracellular T-cell signaling domain nucleic acid sequence. In some cases, a variant or mutant of the CD3ζ intracellular T-cell signaling domain nucleic acid sequence includes substitutions that are conservative substitutions compared to the naturally occurring CD3ζ intracellular T-cell signaling domain nucleic acid sequence.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian (e.g., human) and insect (e.g., spodoptera) cells. Cells may be useful when they are naturally nonadherent or have been treated not to adhere to surfaces, for example by trypsinization.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The terms “plasmid”, “vector” or “expression vector” refer to a nucleic acid molecule that encodes for genes and/or regulatory clements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a peptide compound as described herein and an antigen binding site.

The term “modulation”, “modulate”, or “modulator” are used in accordance with their plain ordinary meaning and refer to the act of changing or varying one or more properties. “Modulator” refers to a composition that increases or decreases the level of a target molecule or the function of a target molecule or the physical state of the target of the molecule. “Modulation” refers to the process of changing or varying one or more properties. For example, as applied to the effects of a modulator on a biological target, to modulate means to change by increasing or decreasing a property or function of the biological target or the amount of the biological target.

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In some cases, inhibition refers to reduction of a disease or symptoms of disease. Thus, In some cases, inhibition includes, at least in part, partially or totally blocking stimulation, decreasing, preventing, or delaying activation, or inactivating, desensitizing, or down-regulating signal transduction or enzymatic activity or the amount of a protein. In some cases, the amount of inhibition may be 10%, 20%, 30%, 40%, 50%, 60%, 70%, 80%, 90%, 100% or less in comparison to a control in the absence of the antagonist. In some cases, the inhibition is 1.5-fold, 2-fold, 3-fold, 4-fold, 5-fold, 10-fold, or more than the expression or activity in the absence of the antagonist.

Depending on context, the term “activation”, “activate”, “activating” and the like in reference to a protein-activator (e.g. agonist) interaction means positively affecting (e.g. increasing) the activity or function of the relative to the activity or function of the protein in the absence of the activator (e.g. composition described herein). Thus, In some cases, activation may include, at least in part, partially or totally increasing stimulation, increasing or enabling activation, or activating, sensitizing, or up-regulating signal transduction or enzymatic activity or the amount of a protein decreased in a disease. The amount of activation may be 10%, 20%, 30%, 40%, 50%, 60%, 70%, 80%, 90%, 100% or more in comparison to a control in the absence of the agonist. In some cases, the activation is 1.5-fold, 2-fold, 3-fold, 4-fold, 5-fold, 10-fold, or more than the expression or activity in the absence of the agonist.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

Depending on context, the term “biological sample” or “sample” refers to a material or materials obtained from or derived from a subject or patient. In some cases, a biological sample includes sections of tissues such as biopsy and autopsy samples, and frozen sections taken for histological purposes. Non-limiting examples of samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some cases, a biological sample from the subject (e.g., such as blood) has aberrant CD6 expression and/or function compared to a control. In some cases, a biological sample from the subject (e.g., such as blood) has aberrant immune cell activity or growth (e.g., an abnormal number of CD6+ immune cells or CD6+ cells with abnormal activity).

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living member of the animal kingdom suffering from or that may suffer from the indicated disorder. In some cases, the subject is a member of a species that includes individuals who naturally suffer from the disease. In some cases, a subject is a living organism suffering from or prone to a discase or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In some cases, the disease is an autoimmune disease (e.g., Type I Diabetes, Graft-versus-Host Disease). In some cases,

An “autoimmune disease” as used herein refers to a disease or disorder that arises from altered immune reactions by the immune system of a subject, e.g., against substances tissues and/or cells normally present in the body of the subject. Autoimmune diseases include, but are not limited to, arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, Graft-versus-Host Disease, and allergic asthma.

As used herein, an “inflammatory disease” refers to a disease or disorder associated with abnormal or altered inflammation. Inflammation is a biological response initiated by the immune system as part of the healing process in response to a pathogen, damaged cells or tissues or irritants. Chronic inflammation can lead to a variety of diseases. Inflammatory diseases include, but are not limited to, atherosclerosis, allergies, asthma, rheumatoid arthritis, transplant rejection, celiac disease, chronic prostatitis, inflammatory bowel diseases, pelvic inflammatory diseases, and inflammatory myopathies.

The term “associated” or “associated with” in the context of a substance or substance activity or function associated with a disease (e.g., an autoimmune disease) means that the disease is caused by (in whole or in part), or a symptom of the disease is caused by (in whole or in part) the substance or substance activity or function.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In some cases, “treating” refers to treatment of an autoimmune disease.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling path way, or reduce one or more symptoms of a disease or condition). An example of a “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

In some cases, the term “administering” includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. In some cases, administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

### Recombinant Proteins and CAR T-Cells

In an aspect, provided herein are recombinant proteins including the proteins expressed by the isolated nucleic acid provided herein, including embodiments thereof. Thus, in an aspect is provided a recombinant protein, such as a chimeric antigen receptor (CAR), that includes a single chain variable fragment (scFv) targeted to CD6 and a transmembrane domain. In an aspect, provided herein is a scFv targeted to CD6 without a transmembrane domain. In an aspect, provided herein is a cell (e.g., a population of cells, such as a population of immune effector cells) engineered to express a CAR, wherein the CAR comprises an antigen-binding domain, and a transmembrane domain. In some cases, the antigen binding domain comprises an antibody fragment or variant targeted to CD6. In some cases, the antigen-binding domain is a single chain variable fragment (scFv) targeted to CD6. In some cases, the CAR further comprises an intracellular signaling domain. In some cases, the cell that expresses the CAR is a T lymphocyte (a CAR T-cell) or an NK cell. In some cases, the cell that expresses the CAR is a T lymphocyte (a CAR T-cell) or an NK cell. In some cases, the cell is a CD4+ T cell, or a CD8+ T cell. In some cases, the T-cell is a regulatory T-cell (Treg).

In some cases, the antigen-binding domain can comprise CDRs of a monoclonal antibody, variable regions of a monoclonal antibody, and/or antigen binding fragments thereof. In some cases, the fragment can be any number of different antigen binding domains of an antigen-specific antibody. In some cases, the antigen-binding domain comprises a CD6-binding fragment of an anti-CD6 antibody. In some cases, the antigen-binding domain comprises the CDRs of an anti-CD6 antibody. In some cases, the antigen-binding domain comprises the VH and VL chains of an anti-CD6 antibody. In some cases, the antigen-binding domain comprises a scFv that comprises CDRs of an anti-CD6 antibody. In some cases, the antigen-binding domain comprises a scFv that comprises the VH and VL chains of an anti-CD6 antibody. In some cases, the anti-CD6 antibody is a monoclonal antibody. In some cases, the monoclonal antibody is a human or a humanized monoclonal antibody. In some cases, the monoclonal antibody is itolizumab. In some cases, the fragment is an antigen-specific scFv encoded by a sequence that is optimized for human codon usage for expression in human cells. In some cases, the monoclonal antibody is a chimericmonoclonal antibody. In some cases, the monoclonal antibody is itolizumab. In some cases, the monoclonal antibody is T12. 1. In some cases, the monoclonal antibody is UMCD6. In some cases, the monoclonal antibody is MEM98. In some cases, the monoclonal antibody is MT605. In some cases, the monoclonal antibody is an antibody described in Gangemi et al. (1989) J Immunol 143 (8): 2439-47 or U.S. Pat. No. 6,572,857, the entire contents of each of whichare incorporated herein by reference. In some cases, the fragment is an antigen-specific scFv encoded by a sequence that is optimized for human codon usage for expression in human cells. In some cases, the antigen-binding domain includes the following VH sequence or a variant thereof: EVOLVESGGGLVKPGGSLKLSCAASGFKFSRYAMSWVRQAPGKRLEWVATISS GGSYIYYP DSVKGRFTISRDNVKNTLYLQMSSLRSEDT AMYYCARRDYDLDYFDSWGQGTLVTVSS (SEQ ID NO: 35). In some cases, the antigen-binding domain includes the following VL sequence or a variant thereof: DIQMTQSPSSLSASVGDRVTITCKASRDIRSYLTWYQQKPGKAPKTLIYYATSLADGVPSRF SGSGSGQDYSLTISSLESDDTATYYCLQHGESPFTFGSGTKLEIKRA (SEQ ID NO: 34). In some cases, the antigen-binding domain includes the following VH sequence or a variant thereof: EVOLVESGGGL VKPGGSLKLSCAASGFKFSRYAMSWVRQTPEKRLEWVATISSGGSYIYYPDSVKGRFTISR DNVKNTLYLQMSSLRSEDTAMYYCARRDYDLDYFDSWGQGTTLTVSS (SEQ ID NO: 68). In some cases, the antigen-binding domain includes the following VL sequence or a variant thercof DIKMTQSPSSMY ASLGERVTITCKASRDIRSYLTWYQQKPWKSPKTLIYYATSLADGVPSRF SGSGSGQDYSLTISSLESDDTATYYCLQHGESPFTFGSGTKLEIKRA (SEQ ID NO:69). In some cases, the VL includes the following sequence or a variant thereof: IQMTQSPSSLSASVGDRVTITCKASRDIRSYLTWYQQKPGKAPKTLIYYATSLADGVPS RFSGSGSGQ (SEQ ID NO:75). In some cases, the VL includes the following sequence or a variant thereof: DIQMTQSPSSLSASVGDRVTITCKASRDIRSY (SEQ ID NO:76). In some cases, the VL includes the following sequence or a variant thereof: LTWYQQKPGKAPKTLIYYATSLADGVPSRFSGSGSGQDYSLTISSLESDDTATYYCLQH GESPFT (SEQ ID NO:77). In some cases, the VL includes the following sequence or a variant thercof: FGSGTKLEIKRA (SEQ ID NO:78). In some cases, the VH includes the following sequence or a variant thereof: EVOLVESGGGLVKPGGSLKLSCAASGFKFSRYAMS (SEQ ID NO: 79). In some cases, the VH includes the following sequence or a variant thereof: WVRQAPGKRLEWVATISSGG (SEQ ID NO:80). In some cases, the VH includes the following sequence or a variant thereof: SYIYYPDSVKGRFTISRDNVKNTLYLQMSSLRSEDTAMYYCARRDYDLDYFDS (SEQ ID NO: 81). In some cases, the VH includes the following sequence or a variant thereof: WGQGTLVTVSS (SEQ ID NO:82). In some cases, a variant of a VH or VL sequence provided hercin has 5, 4, 3, 2, 1, or 0 deletions compared to the sequence. In some cases, a variant of a VH or VL sequence provided herein has 5, 4, 3, 2, 1, or 0 insertions compared to the sequence. In some cases, a variant of a VH or VL sequence provided herein has 5, 4, 3, 2, 1, or 0 substitutions compared to the sequence. Inembodiments, a variant of a VH or VL sequence provided herein has 10, 9, 8, 7, 6, 5, 4, 3, 2, or 1 substitutions compared to the sequence. In some cases, 1 or more or all of the substitutions are conservative substitutions.

TABLE 1
Exemplary Sequences for Antigen
Binding domains and light and
heavy chain variable regions.

| | |
|---|---|
| Heavy CDR1 | VQLVESGGGLVKPGGSLKLS |
| Kabat | (SEQ ID NO: 28) |
| Heavy CDR2 | FSRYAMSWVRQAPGK |
| Kabat | (SEQ ID NO: 29) |
| Heavy CDR3 | RDYDLDYFDSWGQGTLVTV |
| Kabat | (SEQ ID NO: 30) |
| Heavy Chain | EVQLVESGGGLVKPGGSLKLSC |
| Variable | AASGFKFSRYAMSWVRQAPGKR |
| Region | LEWVATISSGGSYIYYPDSVKG |
| | RFTISRDNVKNTLYLQMSSLRS |
| | EDTAMYYCARRDYDLDYFDSWG |
| | QGTLVTVSS |
| | (SEQ ID NO: 35) |
| Light Chain | DIQMTQSPSSLSASVGDRVTIT |
| Variable | CKASRDIRSYLTWYQQKPGKAP |
| Region | KTLIYYATSLADGVPSRFSGSG |
| | SGQDYSLTISSLESDDTATYYC |
| | LQHGESPFTFGSGTKLEIKRA |
| | (SEQ ID NO: 34) |
| Portion of | IQMTQSPSSLSASVGDRVTITC |
| Light Chain | KASRDIRSYLTWYQQKPGKAPK |
| Variable | TLIYYATSLADGVPSRFSGSGS |
| Region | GQ (SEQ ID NO: 93) |

In some cases, the arrangement could be multimeric, such as a diabody or multimers. In some cases, the multimers are most likely formed by cross pairing of the variable portion of the light and heavy chains into what has been referred as a diabody. In some cases, the hinge portion of the construct can have multiple alternatives from being totally deleted, to having the first cysteine maintained, to a proline rather than a serine substitution, to being truncated up to the first cysteine. In some cases, the Fc portion can be deleted. In some cases, any protein that is stable and/or dimerizes can serve this purpose. In some cases, a CAR comprises one of the Fc domains, e.g., either the CH2 or CH3 domain from human immunoglobulin. In some cases, a CAR uses the hinge, CH2 and CH3 region of a human immunoglobulin that has been modified to improve dimerization. In some cases, just the hinge portion of an immunoglobulin is used. In some cases, when expressed, the antigen-binding domain comprises a signal peptide that directs expression of the CAR to the cell membrane. In some cases, the signal peptide is not present in the CAR when it is on the surface of a cell. In some cases, the signal peptide is the same or different from the signal peptide from an antibody that has the CDRs of the antigen-binding domain.

In some cases, the antigen-binding domain (e.g., an scFv that is part of the CAR) has a relatively low affinity (K_{D}) for CD6, while still being specific for CD6. In some cases, the affinity is about 1×10⁻⁴ M to about 1×10⁻⁶ M, about 1×10⁻⁸ M to about 1×10⁻⁷ M, about 1×10⁻⁸ M to about 1×10⁻⁶ M, about 1×10⁻⁹ M to about 1×10⁻⁷ M, about 1.5×10⁻⁸ M to about 1.5×10⁻⁷ M, or about 1×10⁻⁷ M, 2×10⁻⁷ M, 3×10⁻⁷ M, 4×10⁻⁷ M, 5×10⁻⁷ M, 6×10⁻⁷ M, 7×10⁻⁷ M, 8×10⁻⁷ M, 9×10⁻⁷ M, about 1×10⁻⁸ M, 2×10⁻⁸ M, 3×10⁻⁸ M, 4×10⁻⁸ M, 5×10⁻⁸ M, 6×10⁻⁸ M, 7×10⁻⁸ M, 8×10⁻⁸ M, or 9×10⁻⁸ M. One of skill in the art can readily detect the K_{D} of an antibody. In a non-limiting example, affinity may be detected, e.g., by yeast surface display. This approach is a genotype-phenotype linkage strategy mediated by production, secretion, and capture of protein candidates. Candidate proteins can be sorted using combinations of multiple strategies with multiple selection pressures including affinity and stability. See, e.g., Feldhaus et al. (2003) Flow-cytometric isolation of human antibodies from a nonimmune Saccharomyces cerevisiae surface display library. Nature biotechnology. 21:163^{-170;} and Zorniak et al. (207) Yeast display biopanning identifies human antibodies targeting glioblastoma stem-like cells. Scientific Reports. 7:15840, the entire contents of each of which are incorporated herein by reference.

In some cases, an scFv may be designed to have a particular orientation (e.g., V_{H}-V_{L} or V_{L}-V_{H} from the N-terminus to the C-terminus). In some cases, the V_{H} and V_{L} in an scFv are oriented V_{H}-V_{L} from the N-terminus to the C-terminus. In some cases, the V_{H} and V_{L} in an scFv are oriented V_{L}-V_{H} from the N-terminus to the C-terminus. In some cases, the V_{H} and V_{L} portions of the expressed protein will have a slightly different conformation depending on the orientation thereof. In some cases, the orientation of the V_{H} and V_{L} relationship may confer differential affinities for the ligand (e.g., CD6). In some cases, the differential affinity of one orientation compared to another is one order of magnitude or higher.

In some cases, the V_{H} and V_{L} portions of the scFv are directly adjacent and contiguous to one another. Alternatively, the V_{H} and V_{L} of the scFv may be separated by a linker (e.g., flexible linker). In some cases, the linker is a polypeptide linker. In some cases, the polypeptide linker is a flexible linker. In some cases, the V_{H} and V_{L} (in either orientation) of the scFv may be fused through a flexible linker with different sizes e.g.: 18 amino acids, 19 amino acids, or 20 amino acids; allowing the V_{H} and V_{L} of the scFv to fold into their native configuration and conserving the antigen-binding properties. In some cases, the linker includes glycine and/or serine residues. In some cases, the linker has the amino acid sequence GSTSGGGSGGGSGGGGSS (SEQ ID NO:36). In some cases, the linker has the amino acid sequence GGGGSGGGGSGGGGSGGGGS (SEQ ID NO: 37). In some cases, the linker is about 18 amino acids in length. In some cases, the linker is 18 amino acids in length. In some cases, the linker is about 19 amino acids in length. In some cases, the linker is 19 amino acids in length. In some cases, the linker is about 20 amino acids in length. In some cases, the linker is 20 amino acids in length. In some cases, the linker sequence is long enough to allow the expressed scFv to fold into its native configuration. In some cases, the linker sequence is long enough to allow the expressed scFv to maintain its antigen-binding properties. Techniques for assessing protein folding include, but are not limited to, NMR, X-Ray crystallography, circular dichroism, fluorescence spectroscopy, dual polarization interferometry, and other techniques well known in the art. Techniques for assessing antigen-binding behavior include, but are not limited to, surface plasm on resonance (SPR), various ligand binding assays, and other techniques well known in the art.

In some cases, a recombinant protein provided herein, including embodiments thereof, may further include additional components of a CAR. CARs that include such additional components and cells expressing such CARs are also included.

In some cases, the recombinant protein (such as a CAR) provided herein (e.g. isolated or expressed on the surface of a cell), including embodiments thereof, may further include a transmembrane domain as described herein, including embodiments thereof, a hinge region as described herein, including embodiments thereof, an intracellular signaling domain (such as a T-cell signaling domain) as described herein, including embodiments thereof, and/or a co-stimulatory domain as described herein, including embodiments thereof.

In some cases, an scFv as provided herein may be directly connected (e.g., covalently bound) to a transmembrane domain or may be connected (e.g., covalently bound) to the transmembrane domain through a hinge region.

A “hinge region” as provided herein is a polypeptide connecting an antigen-binding site domain (i.e., scFv) to a transmembrane domain. Any hinge region capable of connecting an scFv to a transmembrane domain is contemplated herein. In some cases, the hinge region is a polypeptide hinge region. In some cases, the polypeptide hinge region is a flexible hinge region. In some cases, the hinge region includes glycine and/or serine residues. In some cases, the hinge region is or includes an antibody hinge region or a portion thereof. In some cases, the hinge region includes an antibody Fc domain or a portion thereof (e.g., a portion comprising a hinge region). Non-limiting examples of suitable hinge regions contemplated herein include a IgG Fc, human IgG Fc, human IgG1 Fc, IgG4 Fc, and human IgG4 Fc, or a portion thereof (i.e., a portion thereof that includes the hinge region). In some cases, the hinge region is an IgG Fc or a portion thereof. In some cases, the hinge region is a human IgG Fc or a portion thereof. In some cases, the hinge region is an IgG1 Fc or a portion thereof. In some cases, the hinge region is a human IgG1 Fc, or a portion thereof. In some cases, the hinge region is an IgG4 Fc, or a portion thereof. In some cases, the hinge regions is an human IgG4 Fc, or a portion thereof. In some cases, a IgG Fc hinge is used to connect the binding site domain to the CAR backbone. In some cases, an IgG1 or IgG4 human Fc or a portion thereof comprising a hinge region include one or more mutations to reduce or prevent the binding to the corresponding Fc receptor. In some cases, an IgG1 or IgG4 human Fc or a portion thereof comprising a hinge region does not include a mutation to reduce or prevent the binding to the corresponding Fc receptor. In some cases, the length of a portion of a Fc domain can be of 229 amino acids, 129 amino acids, or less.

In some cases, the hinge region is about 229 amino acids in length. In some cases, the hinge region is 229 amino acids in length. In some cases, the hinge region is about 129 amino acids in length. In some cases, the hinge region is 129 amino acids in length. In some cases, the hinge region is between about 129 to about 229 amino acids in length. In some cases, the hinge region is less than about 229 amino acids in length. In some cases, the hinge region is less than 229 amino acids in length. In some cases, the hinge region is less than about 129 amino acids in length. In some cases, the hinge region is less than 129 amino acids in length. In some cases, the hinge region is at least 22 amino acids in length. In some cases, the hinge region is 22 amino acids in length. In some cases, the hinge region is about 25, 50, 75, 100, 125, 150, 175, 200, 225, or 250 amino acids in length. In some cases, the hinge region is about 25 amino acids in length. In some cases, the hinge region is about 50 amino acids in length. In some cases, the hinge region is about 75 amino acids in length. In some cases, the hinge region is about 100 amino acids in length. In some cases, the hinge region is about 125 amino acids in length. In some cases, the hinge region is about 150 amino acids in length. In some cases, the hinge region is about 175 amino acids in length. In some cases, the hinge region is about 200 amino acids in length. In some cases, the hinge region is about 225 amino acids in length. In some cases, the hinge region is about 250 amino acids in length. In some cases, the hinge region is 25 amino acids in length. In some cases, the hinge region is 50 amino acids in length. In some cases, the hinge region is 75 amino acids in length. In some cases, the hinge region is 100 amino acids in length. In some cases, the hinge region is 125 amino acids in length. In some cases, the hinge region is 150 amino acids in length. In some cases, the hinge region is 175 amino acids in length. In some cases, the hinge region is 200 amino acids in length. In some cases, the hinge region is 225 amino acids in length. In some cases, the hinge region is 250 amino acids in length.

In some cases, the hinge region is between about 22 to about 250 amino acids in length. In some cases, the hinge region is between about 25 to about 250 amino acids in length. In some cases, the hinge region is between about 50 to about 250 amino acids in length. In some cases, the hinge region is between about 75 to about 250 amino acids in length. In some cases, the hinge region is between about 100 to about 250 amino acids in length. In some cases, the hinge region is between about 125 to about 250 amino acids in length. In some cases, the hinge region is between about 150 to about 250 amino acids in length. In some cases, the hinge region is between about 175 to about 250 amino acids in length. In some cases, the hinge region is between about 200 to about 250 amino acids in length. In some cases, the hinge region is between about 225 to about 250 amino acids in length. In some cases, the hinge region is between about 22 to about 225 amino acids in length. In some cases, the hinge region is between about 22 to about 200 amino acids in length. In some cases, the hinge region is between about 22 to about 175 amino acids in length. In some cases, the hinge region is between about 22 to about 150 amino acids in length. In some cases, the hinge region is between about 22 to about 125 amino acids in length. In some cases, the hinge region is between about 22 to about 100 amino acids in length. In some cases, the hinge region is between about 22 to about 75 amino acids in length. In some cases, the hinge region is between about 22 to about 50 amino acids in length. In some cases, the hinge region is between about 22 to about 25 amino acids in length.

In some cases, the hinge region includes an Fc domain. In some cases, the hinge region includes an IgG Fc domain. In some cases, the hinge region is an IgG1 Fc or a fragment thereof. In some cases, the hinge region is a human IgG1 Fc or a fragment thereof. In some cases, the hinge region is an IgG4 Fc or a fragment thereof. In some cases, the hinge region is a human IgG4 Fc or a fragment thereof.

In some cases, the hinge region may be a mutated hinge region. In some cases, a mutated hinge region (e.g., Fc) may be useful, for example, to prevent the Fc from binding to its cognate Fc receptor.In some cases, the hinge region (e.g., IgG, human IgG Fc, IgG1 Fc, human IgG1 Fc, IgG4 Fc, human IgG4 Fc) includes no more than 5, 4, 3, 2, or 1 deletions. In some cases, the hinge region (e.g., IgG, human IgG Fc, IgG1 Fc, human IgG1 Fc, IgG4 Fc, human IgG4 Fc) includes no more than 5, 4, 3, 2, or 1 insertions. In some cases, the hinge region (e.g., IgG, human IgG Fc, IgG1 Fc, human IgG1 Fc, IgG4 Fc, human IgG4 Fc) does not include deletions. In some cases, the hinge region (e.g., IgG, human IgG Fc, IgG1 Fc, human IgG1 Fc, IgG4 Fc, human IgG4 Fc) does not include insertions. In some cases, the hinge region (e.g., IgG, human IgG Fc, IgG1 Fc, human IgG1 Fc, IgG4 Fc, human IgG4 Fc) includes substitutions that are conservative substitutions.

In some cases, a transmembrane domain as provided herein refers to a polypeptide forming part of (e.g., spanning) a biological membrane. In some cases, the transmembrane domain provided herein is capable of spanning a biological membrane (e.g., a cellular membrane) from one side of the membrane through to the other side of the membrane. In some cases, the transmembrane domain spans only a portion of the membrane, but is sufficient to anchor an antigen-binding domain to the membrane. In some cases, the transmembrane domain spans from the intracellular side to the extracellular side of a cellular membrane. In some cases, transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof is contemplated. Non-limiting examples of transmembrane domains include the transmembrane domains of CD28, CD8, CD4, or CD3-zeta (CD3ζ).

In some cases, the transmembrane domain includes a CD4 transmembrane domain or a variant thereof, a CD8 transmembrane domain or a variant thereof, a CD28 transmembrane domain or a variant thereof, or a CD3ζ transmembrane domain or a variant thereof. In some cases, recombinant protein such as a CAR (e.g., a cell expressing a CAR) comprises an intracellular co-stimulatory domain and/or an intracellular T-cell signaling domain.

In some cases, the transmembrane domain includes a CD4 transmembrane domain or a variant thereof. In some cases, the transmembrane domain includes a CD8 transmembrane domain or a variant thereof. In some cases, the transmembrane domain includes a CD28 transmembrane domain or a variant thereof. In some cases, the transmembrane domain includes a CD3ζ transmembrane domain or a variant thereof.

In some cases, the transmembrane domain is covalently bound to a heavy chain variable region of the scFv. In some cases, the transmembrane domain is covalently bound to a light chain variable region of the scFv. In some cases, the transmembrane domain is covalently bound to a heavy chain variable region of the scFv though a hinge region. In some cases, the transmembrane domain is covalently bound to a light chain variable region of the scFv through a hinge region. In some cases, a recombinant protein provided herein (such as a CAR, e.g., on the surface of a cell) comprises, from the N-terminal end to the C-terminal end a V_{H}, a V_{L}, and a transmembrane domain. In some cases, a recombinant protein provided herein (such as a CAR, e.g., on the surface of a cell) comprises, from the N-terminal end to the C-terminal end a V_{L}, a V_{H}, and a transmembrane domain. In some cases, a recombinant protein provided herein (such as a CAR, e.g., on the surface of a cell) comprises, from the N-terminal end to the C-terminal end a V_{H}, a linker, a V_{L}, and a transmembrane domain. In some cases, a recombinant protein provided herein (such as a CAR, e.g., on the surface of a cell) comprises, from the N-terminal end to the C-terminal end a V_{L}, a linker, a V_{H}, and a transmembrane domain. In some cases, a recombinant protein provided herein (such as a CAR, e.g., on the surface of a cell) comprises, from the N-terminal end to the C-terminal end a V_{H}, a linker, a V_{L}, a hinge region, and a transmembrane domain. In some cases, a recombinant protein provided herein (such as a CAR, e.g., on the surface of a cell) comprises, from the N-terminal end to the C-terminal end a V_{L}, a linker, a V_{H}, a hinge region, and a transmembrane domain. The VH, VL, linker, hinge region, and transmembrane domain referred to in this paragraph, refer to those disclosed herein, including embodiments thercof.

In some cases, the antigen-binding domain (e.g., the scFv) includes the CDR sequences of itolizumab. In some cases, the CDRs include the amino acid sequences set forth by SEQ ID NOs: 28, 29, 30, 31, 32, and 33. In some cases, the CDRs are the amino acid sequences set forth by SEQ ID NOs: 28, 29, 30, 31, 32, and 33. In some cases, the CDRs specifically bind CD6. In some cases, the CDRs specifically bind CD6 with an affinity from between about 1×10⁻⁶ to 1×10⁻⁸. In some cases, the CDRs specifically bind CD6 with an affinity of equal to or less than about 1×10⁻⁶ or 1×10⁻⁷.

An “intracellular co-stimulatory signaling domain,” “co-stimulatory signaling domain,” or “intracellular co-stimulatory domain” as provided herein includes amino acid sequences capable of providing co-stimulatory signaling in response to binding of an antigen to the antigen-binding domain of the recombinant protein (e.g. CAR) provided herein including embodiments thereof. In some cases, the signaling of the co-stimulatory signaling domain results in production of cytokines and proliferation of a cell (e.g., a T cell) expressing the recombinant protein (e.g., CAR).

In some cases, the co-stimulatory domain is a CD28 intracellular co-stimulatory domain or a variant thereof, a 4⁻¹BB intracellular co-stimulatory domain or a variant thereof, an ICOS intracellular co-stimulatory signaling domain or a variant thereof, an OX-40 intracellular co-stimulatory signaling domain or a variant thereof, a CTLA-4 intracellular co-stimulatory domain or a variant thereof, a PD-1 intracellular co-stimulatory domain or a variant thereof, or a GITR co-stimulatory domain or a variant thereof. In some cases, the co-stimulatory domain is a CD28 intracellular co-stimulatory domain (SEQ ID NO:7 or 8) or a variant thereof. In some cases, a 4-1 BB intracellular co-stimulatory domain (SEQ ID NO: 14) or a variant thereof. In some cases, the co-stimulatory domain is an ICOS intracellular co-stimulatory signaling domain or a variant thereof. In some cases, the co-stimulatory domain is an OX-40 intracellular co-stimulatory signaling domain or a variant thereof. In some cases, the co-stimulatory domain is a CTLA-4 intracellular co-stimulatory domain (SEQ ID NO:17) or a variant thereof. In some cases, the co-stimulatory domain is a PD-1 intracellular co-stimulatory domain or a variant thereof. In some cases, the co-stimulatory domain is a GITR co-stimulatory domain or a variant thereof.

In some cases, a recombinant protein provided herein (such as a CAR, e.g., on the surface of a cell) comprises, from the N-terminal end to the C-terminal end a V_{H}, a V_{L}, a transmembrane domain, and a co-stimulatory domain. In some cases, a recombinant protein provided herein (such as a CAR, e.g., on the surface of a cell) comprises, from the N-terminal end to the C-terminal end a V_{L}, a V_{H}, a transmembrane domain, and a co-stimulatory domain. In some cases, a recombinant protein provided herein (such as a CAR, e.g., on the surface of a cell) comprises, from the N-terminal end to the C-terminal end a V_{H}, a linker, a V_{L}, a transmembrane domain, and a co-stimulatory domain. In some cases, a recombinant protein provided herein (such as a CAR, e.g., on the surface of a cell) comprises, from the N-terminal end to the C-terminal end a V_{L}, a linker, a V_{H}, a transmembrane domain, and a co-stimulatory domain. In some cases, a recombinant protein provided herein (such as a CAR, e.g., on the surface of a cell) comprises, from the N-terminal end to the C-terminal end a V_{H}, a linker, a V_{L}, a hinge region, a transmembrane domain, and a co-stimulatory domain. In some cases, a recombinant protein provided herein (such as a CAR, e.g., on the surface of a cell) comprises, from the N-terminal end to the C-terminal end a V_{L}, a linker, a V_{H}, a hinge region, a transmembrane domain, and a co-stimulatory domain. The VH, VL, linker, hinge region, transmembrane domain, and a co-stimulatory domain referred to in this paragraph, refer to those disclosed herein, including embodiments thereof.

An “intracellular T-cell signaling domain” as provided herein includes amino acid sequences capable of providing primary signaling in response to binding of an antigen to the antigen-binding domain of the recombinant protein (e.g. CAR) provided herein including embodiments thereof, including embodiments thereof. In some cases, the signaling of the intracellular T-cell signaling domain results in activation of the T cell expressing the same. In some cases, the signaling of the intracellular T-cell signaling domain results in proliferation (cell division) of a cell (e.g., a T cell) expressing the recombinant protein (e.g., CAR). In some cases, the signaling of the intracellular T-cell signaling domain results in expression by the T cell of proteins known in the art to characteristic of activated T cell (e.g., CTLA-4, PD-1, CD28, CD69).

In some cases, the intracellular T-cell signaling domain is a CD3ζ intracellular T-cell signaling domain.

In some cases, a recombinant protein provided herein (such as a CAR, e.g., on the surface of a cell) comprises, from the N-terminal end to the C-terminal end a V_{H}, a V_{L}, a transmembrane domain, a co-stimulatory domain, and an intracellular T-cell signaling domain. In some cases, a recombinant protein provided herein (such as a CAR, e.g., on the surface of a cell) comprises, from the N-terminal end to the C-terminal end a V_{L}, a V_{H}, a transmembrane domain, a co-stimulatory domain, and an intracellular T-cell signaling domain. In some cases, a recombinant protein provided herein (such as a CAR, e.g., on the surface of a cell) comprises, from the N-terminal end to the C-terminal end a V_{H}, a linker, a V_{L}, a transmembrane domain, a co-stimulatory domain, and an intracellular T-cell signaling domain. In some cases, a recombinant protein provided herein (such as a CAR, e.g., on the surface of a cell) comprises, from the N-terminal end to the C-terminal end a V_{L}, a linker, a V_{H}, a transmembrane domain, a co-stimulatory domain, and an intracellular T-cell signaling domain. In some cases, a recombinant protein provided herein (such as a CAR, e.g., on the surface of a cell) comprises, from the N-terminal end to the C-terminal end a V_{H}, a linker, a V_{L}, a hinge region, a transmembrane domain, a co-stimulatory domain, and an intracellular T-cell signaling domain. In some cases, a recombinant protein provided herein (such as a CAR, e.g., on the surface of a cell) comprises, from the N-terminal end to the C-terminal end a V_{L}, a linker, a V_{H}, a hinge region, a transmembrane domain, a co-stimulatory domain, and an intracellular T-cell signaling domain. The VH, VL, linker, hinge region, transmembrane domain, co-stimulatory domain, and intracellular T-cell signaling domain referred to in this paragraph, refer to those disclosed herein, including embodiments thereof.

In some cases, the T lymphocyte is a regulatory T lymphocyte (Treg). The category of effector T cell is a broad one that includes various T cell types that actively respond to a stimulus, such as co-stimulation. Effector T cells include helper, killer, regulatory, and potentially other T cell types. The regulatory T cells, formerly known as suppressor T cells, are a subpopulation of T cells which modulate the immune system, maintain tolerance to self-antigens, and prevent autoimmune disease. Tregs are immunosuppressive and generally suppress or downregulate induction and proliferation of effector T cells. In some cases, the effector T lymphocyte helper T cell. In some cases, the effector T lymphocyte is a killer T cell. In some cases, the effector T lymphocyte is a Treg.

In some cases, the Treg is a Treg that includes CD4positive-CD25high expression. In some cases, the Treg is a Treg that includes CD4positive-CD25high-CD127low or negative expression. In some cases, the Treg is a Treg that includes CD4positive-CD25high-CD6low or negative expression. In some cases, the Treg is a Treg that includes CD4positive-CD25high-CD127low or negative-CD6low or negative expression. In some cases, the Treg is a Treg that includes CD3positive-CD6low or negative expression. In some cases, the Treg is a Treg that includes CD4positive-CD6low or negative expression. In some cases, the Treg is a Treg that includes CD8positive-CD28low expression. In some cases, the Treg is a Treg that includes CD45RA+ expression. In some cases, the Treg is a Treg that does not include CD45RA+ expression. In some cases, the Treg is a Treg that includes FOXP3 demethylation. In some cases, the Treg is a Treg that does not include FOXP3 demethylation. In some cases, the Treg is a Treg that can be expanded either with IL-2 and/or rapamycin and/or retinoic acid. In some cases, the Treg is a Treg that can be expanded with IL-2 and/or rapamycin and/or retinoic acid. In some cases, the Treg is a Treg that can be expanded with IL-2. In some cases, the Treg is a Treg that can be expanded with rapamycin. In some cases, the Treg is a Treg that can be expanded with retinoic acid.

Determining “high” and “low” expression is well known in the art. A description of high and low expression of the markers referred to supra, and how high and low expression are determined and quantified, may be found, for example, in Putnam, A. L., T. M. Brusko, M. R. Lee, W. Liu, G. L. Szot, T. Ghosh, M. A. Atkinson and J. A. Bluestone. Expansion of human regulatory T-cells from patients with type 1 diabetes. Diabetes 58(3): 652-662, 2009; Bluestone J A, Buckner J H, Fitch M, Gitelman S E, Gupta S, Hellerstein M K, Herold K C, Lares A, Lec M R, Li K, Liu W, Long S A, Masiello L M, Nguyen V, Putnam AL, Rieck M, Sayre PH, Tang Q. Type 1 diabetes immunotherapy using polyclonal regulatory T cells. Sci Transl Med. 2015 Nov. 25;7(315):315ra189; Fuchs, A., M. Gliwinski, N. Grageda, R. Spiering, A. K. Abbas, S. Appel, R. Bacchetta, M. Battaglia, D. Berglund, B. Blazar, J. A. Bluestone, M. Bornhauser, A. Ten Brinke, T. M. Brusko, N. Cools, M. C. Cuturi, E. Geissler, N. Giannoukakis, K. Golab, D. A. Hafler, S. M. van Ham, J. Hester, K. Hippen, M. Di lanni, N. Ilic, J. Isaacs, F. Issa, D. Iwaszkiewicz-Grzes, E. Jaeckel, I. Joosten, D. Klatzmann, H. Koenen, C. van Kooten, O. Korsgren, K. Kretschmer, M. Levings, N. M. Marek-Trzonkowska, M. Martinez-Llordella, D. Miljkovic, K. H. G. Mills, J. P. Miranda, C. A. Piccirillo, A. L. Putnam, T. Ritter, M. G. Roncarolo, S. Sakaguchi, S. Sanchez-Ramon, B. Sawitzki, L. Sofronic-Milosavljevic, M. Sykes, Q. Tang, M. Vives-Pi, H. Waldmann, P. Witkowski, K. J. Wood, S. Gregori, C. M. U. Hilkens, G. Lombardi, P. Lord, E. M. Martinez-Caceres and P. Trzonkowski. Minimum Information about T Regulatory Cells: A Step toward Reproducibility and Standardization. Front Immunol 8: 1844, 2017; Duggleby, R., R. D. Danby, J. A. Madrigal and A. Saudemont. Clinical Grade Regulatory CD4(+) T Cells (Tregs): Moving Toward Cellular-Based Immunomodulatory Therapies. Front Immunol 9:252, 2018; the entire contents of each of which are incorporated herein by reference in their entireties and for all purposes.

In some cases, an scFv (e.g., of a CAR) includes a light chain variable region set forth by SEQ ID NO:34. In some cases, the scFv includes a heavy chain variable region set forth by SEQ ID NO:35. In some cases, the scFv includes the sequence set forth by SEQ ID NO:38. In some cases, the scFv includes the sequence set forth by SEQ ID NO:39.

In some cases, an scFv (e.g., of a CAR) is oriented with a light chain variable region at the N-terminus followed by a heavy chain variable region. Alternatively, In some cases, the scFv is oriented with the heavy chain variable region at the N-terminus followed by the light chain variable region.

In some cases, the light chain variable region (VL) and the heavy chain variable region (VH) of the scFv are separated by a linker. In some cases, the linker comprises the sequence set forth by SEQ ID NO:36. In some cases, the linker comprises the sequence set forth by SEQ ID NO: 37.

In some cases, the scFv amino acid sequence includes the sequence set forth by SEQ ID NO: 38. In some cases, the scFv amino acid sequence includes the sequence set forth by SEQ ID NO: 39. In some cases, the scFv amino acid sequence includes the sequence set forth by SEQ ID NO: 40. In some cases, the scFv amino acid sequence includes the sequence set forth by SEQ ID NO: 41.

In some cases, the light chain variable region is covalently bound to the transmembrane region through a hinge region. In some cases, the heavy chain variable region is covalently bound to the transmembrane region through a hinge region.

In some cases, the hinge region is a human IgG Fc. In some cases, the human IgG Fc is a human IgG4 Fc. In some cases, the human IgG Fc is a human IgG1 Fc.

In some cases, the co-stimulatory domain is a CD28 intracellular co-stimulatory domain or a variant thereof, a 4⁻¹BB intracellular co-stimulatory domain or a variant thereof, an ICOS intracellular co-stimulatory signaling domain or a variant thereof, an OX-40 intracellular co-stimulatory signaling domain or a variant thereof, a CTLA-4 intracellular co-stimulatory signaling domain or a variant thereof, a PD-1 intracellular co-stimulatory signaling domain or a variant thereof, or a GITR intracellular co-stimulatory signaling domain or a variant thereof.

In some cases, the intracellular T-cell signaling domain is a CD3ζ intracellular T-cell signaling domain.

In some cases, the scFv comprises the CDR sequences set forth by SEQ ID NOs:28, 29, 30, 31, 32, and 33.

In an aspect is provided a T lymphocyte including the recombinant protein described herein including embodiments thereof. In some cases, the T lymphocyte is a regulatory T lymphocyte (Treg) as described herein, including embodiments thereof. CAR T cells may be produced using methods well known in the art. For example, T lymphocytes isolated from a subject (e.g. patient) or donor (e.g. healthy subject) may be transduced with a nucleic acid encoding a CAR. Introduction of the nucleic acid encoding the CAR may be accomplished by viral or non-viral methods as described surpa. It should be appreciated that T lymphocytes obtained from donors (i.e. allogenic T lymphocytes) may undergo gene editing, using gene editing techniques well known in the art (e.g. CRISPR), to eliminate immunogenic proteins (e.g. native T cell receptors). Following transduction, the T lymphocytes may be activated using, for example, artificial antigen-presenting cells (APCs; e.g. engineered cell lines or antibody-coated magnetic beads). Once activated, the T lymphocytes may be induced to develop into specialized T cell subtypes (e.g. T regulatory cells) by treating with, for example, specific mixtures of cytokines. Finally, the CAR T cell population may be expanded using techniques well known in the art.

In some cases, the T lymphocyte including the recombinant protein described herein, including embodiments thereof, is an autologous T lymphocyte (i.e. taken from the subject). In some cases, the T lymphocyte including the recombinant protein described herein, including embodiments thereof, is an allogenic T lymphocyte (i.e. a T lymphocyte not obtained from the subject.) In some cases, the allogenic T lymphocyte has undergone gene editing. In some cases, the allogeneic T lymphocyte is gene edited to eliminate expression of a native T cell receptor protein.

In an aspect, an isolated nucleic acid encoding a protein (e.g. a CAR) including a single chain variable fragment (scFv) targeted to CD6 and a transmembrane domain is provided.

In an aspect, a vector including the nucleic acid (e.g., isolated nucleic acid) as provided herein including embodiments thereof is provided. In some cases, the vector is a composition of matter that comprises an isolated nucleic acid and that can be used to deliver the isolated nucleic acid to the interior of a cell. Numerous vectors are known in the art including, but not limited to, linear polynucleotides, polynucleotides associated with ionic or amphiphilic compounds, plasmids, and viruses. In some cases, the vector is an autonomously replicating plasmid or a virus. In some cases, a compound that facilitates transfer of nucleic acid into cells, such as, for example, a polylysine compound, liposome, and the like is used. Examples of viral transfer vectors include, but are not limited to, adenoviral vectors, adeno-associated virus vectors, retroviral vectors, lentiviral vectors, and the like. In some cases, the vector is a plasmid. In some cases, the vector is extrachromosomal. In some cases, the vector integrates into the genome of a cell. In some cases, the vector is a viral vector. In some cases, the virus is a lentivirus or onco-retrovirus. In some cases, the virus is a lentivirus. In some cases, the virus is an onco-retrovirus. Any suitable virus for delivery of a vector including the nucleic acid provided herein (e.g., the isolated nucleic acid) to a cell is contemplated In some cases, the vector comprises a recombinant polynucleotide comprising expression control sequences operatively linked to the nucleotide sequence to be expressed. The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAXTM vector system from Lentigen and the like. Nonclinical types of lentiviral vectors are also available and would be known to one skilled in the art.

In an aspect is provided a T lymphocyte including a vector provided herein, including embodiments thereof.

### Methods of Treatment

The compositions provided herein, including embodiments thereof, are contemplated as effective treatment of autoimmune diseases. Thus, in an aspect is provided a method of treating an autoimmune disease (e.g., Type I diabetes, Graft-versus-Host Disease, Lupus), the method including administering to a subject in need thereof an effective amount of a T-lymphocyte (e.g., a CART-cell) provided herein including embodiments thereof.

In some cases, the autoimmune disease is associated with reduced islet cell (e.g., beta cell) function, viability, or survival. In some cases, the autoimmune disease is Type I Diabetes. In some cases, the autoimmune disease is Graft-versus-Host Disease. In some cases, the autoimmune disease includes a subject's immune system attacking the subject's islet cells (e.g., beta cells).

In some cases, the T-Lymphocyte provided herein, including embodiments thereof, suppresses CD6+ T-lymphocytes. In some cases, the T-Lymphocyte provided herein, including embodiments thereof, suppresses CD6+ B-lymphocytes.

### EXAMPLES

The following examples are intended to further illustrate certain embodiments of the disclosure. The examples are put forth so as to provide one of ordinary skill in the art and are not intended to limit its scope.

### Example 1: CD6-Targeted CAR Expressed in Treg

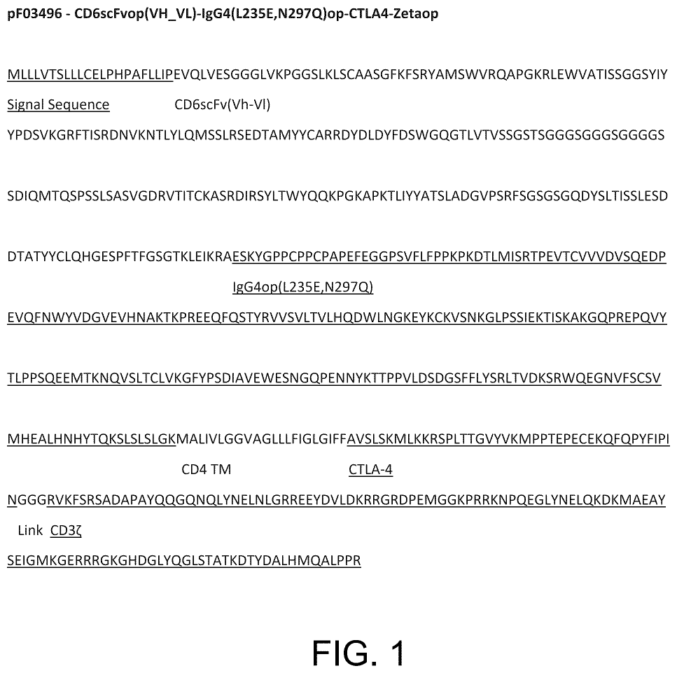

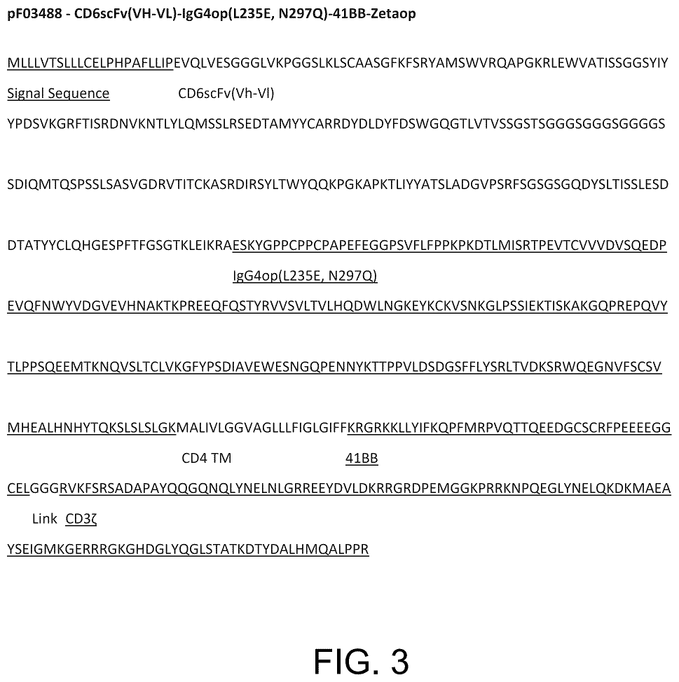

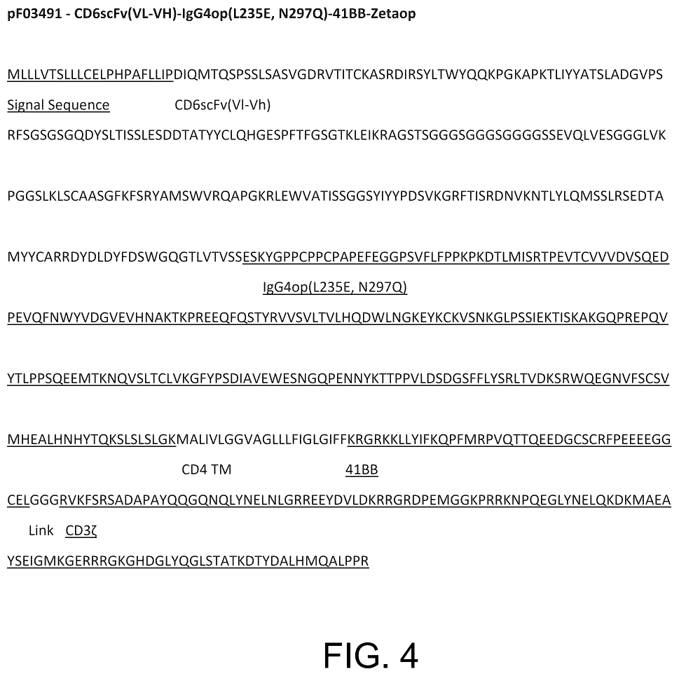

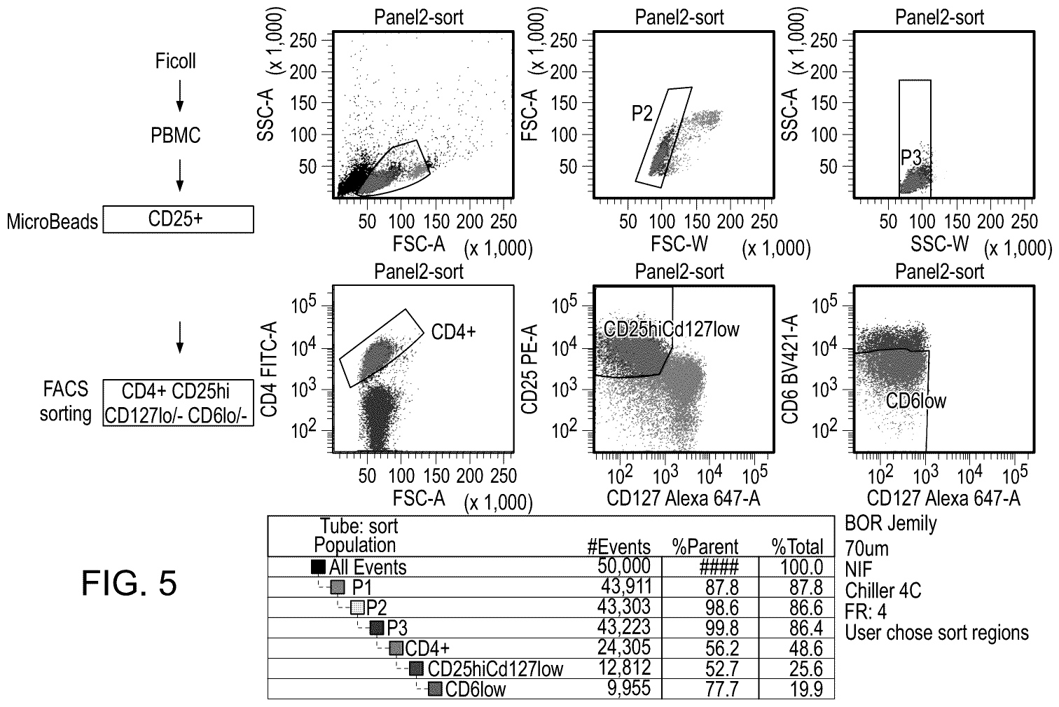

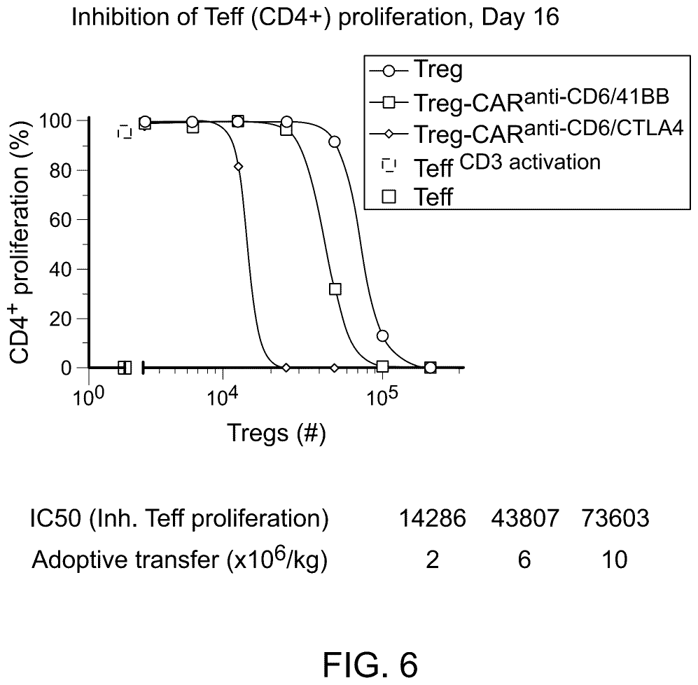

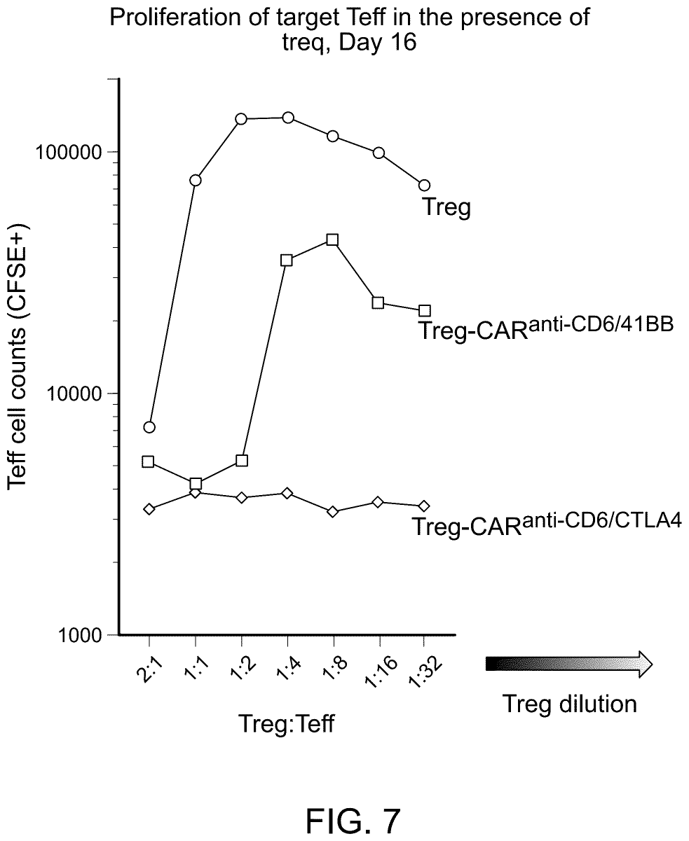

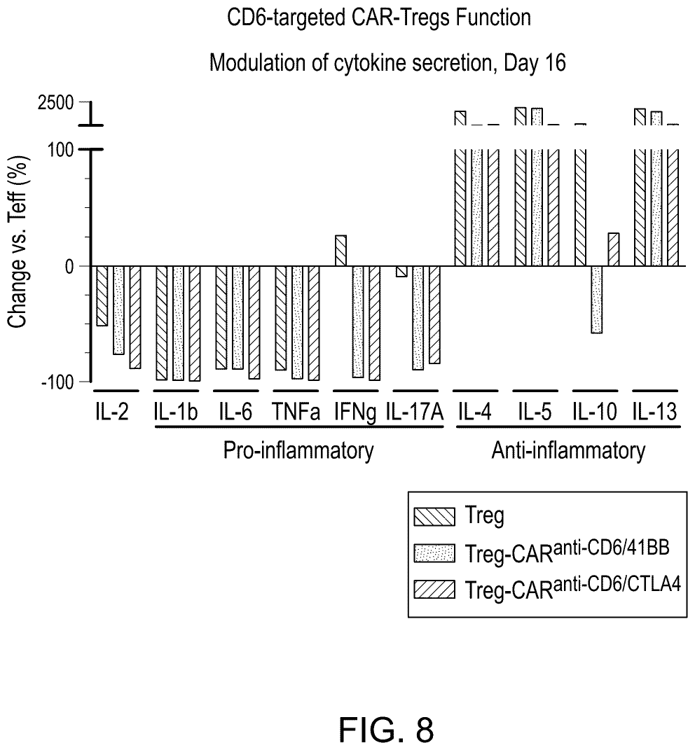

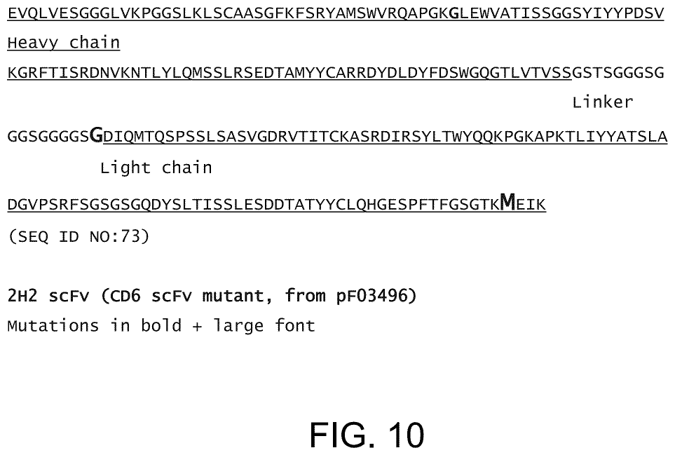

Various CAR that include an scFv derived from Itolizumab are depicted in FIG. 1 to FIG. 4. Each includes, in addition to an scFv in either the VL or VH orientation: a spacer derived from a portion of IgG4 with certain mutations, a CD4 transmembrane domain, either a CTLA4 or 4-1BB signaling domain, and a CD3 zeta signaling domain. CAR were prepared with the scFv in VH-VL or VL-VH orientations because yeast surface display technology previously suggested that a VH-VL scFv has an affinity for human CD6 that is similar to Itolizumab while a VL-VH scFv has lower affinity for human CD6 (Garner et al. (2018) Immunology 155:273). FIG. 5 is schematic depiction of a method used to isolate Tregs and Tregs that are CD6^{low/-}. Tregs can be isolated using any appropriate method (Fuchs et al. (2018) Front Immunol. 8:1844; Duggleby et al.) 2018) Front Immunol. 9:252). A comparison of Treg, Treg expressing a CD6 CAR (CTLA4), and a CD6 CAR (4-1BB) shows that the CD6 CAR with CTLA4 signaling domain is superior for reducing Teff (CD4+) proliferation (FIG. 6). In addition, the CD6 CAR with CTLA4 signaling domain is superior for reducing target Teff proliferation (FIG. 7). The Treg expressing CD6 elicited anti-inflammatory cytokines (FIG. 8). As shown in FIG. 10, Tregs expressing a CD6 CAR with a CTLA4 signaling domain can be cultured in the presence of Teff while expressing relatively low levels of exhaustion markers.

### Example 2: Expression of CD6-Targeted CAR

The CD6-targeted CAR can expressed in Tregs using a lentiviral vector. A suitable lentiviral vector is described in WO 2016/044811.The nucleotide sequence expressing the CAR can be in frame with a sequence encoding T2A (LEGGGEGRGSLLTCGDVEENPGPR; SEQ ID NO: 71) and a sequence encoding a truncated CD19 receptor (MPPPRLLFFLLFLTPMEVRPEEPLVVKVEEGDNAVLQCLKGTSDGPTQQLTWSRESPLKPF LKLSLGLPGLGIHMRPLAIWLFIFNVSQQMGGFYLCQPGPPSEKAWQPGWTVNVEGSGELF RWNVSDLGGLGCGLKNRSSEGPSSPSGKLMSPKLYVWAKDRPEIWEGEPPCVPPRDSLNQS LSQDLTMAPGSTLWLSCGVPPDSVSRGPLSWTHVHPKGPKSLLSLELKDDRPARDMWVME TGLLLPRATAQDAGKYYCHRGNLTMSFHLEITARPVLWHWLLRTGGWKVSAVTLAYLIFC LCSLVGILHLQRALVLRRKR; SEQ ID NO:73). When expressed in this manner, the CAR is coordinately expressed with a truncated CD19. This facilitates quantification of CAR-expressing cells using readily available CD19 antibodies.

FIG. 10 depicts the amino acid sequence of an alternative CD6 scFv that can replace the scFv in any of the constructs depicted on FIGS. 1-4.

What is claimed is:

1. An isolated nucleic acid encoding a chimeric antigen receptor (CAR) comprising a single chain variable fragment (scFv) targeted to CD6, a hinge region, a transmembrane domain, a CTLA4 signaling domain and a CD3 zeta signaling domain.

2. The isolated nucleic acid of claim 1, wherein said transmembrane domain comprises a CD4 transmembrane domain or a variant thereof, a CD8 transmembrane domain or a variant thereof, a CD28 transmembrane domain or a variant thereof, or a CD3ζ transmembrane domain or a variant thereof.

3. The isolated nucleic acid of claim 1, wherein the CAR comprises or consists of an amino acid sequence at least 95% identical to any of SEQ ID Nos: 83-8.

4. The isolated nucleic acid of claim 1, wherein the CAR comprises or consists of an amino acid sequence of any of SEQ ID Nos: 83-86 with no more than 5 single amino acid substitutions.

5. The isolated nucleic acid of claim 1, wherein said scFv comprises a light chain variable region (VL) comprising the amino acid sequence set forth by SEQ ID NO:34.

6. The isolated nucleic acid of claim 1, wherein said scFv comprises a heavy chain variable region (VH) comprising the amino acid sequence set forth by SEQ ID NO:35.

7. The isolated nucleic acid of claim 1, wherein said scFv comprises a light chain variable region (VL) complementarity determining region (CDR) 1 (VL-CDR1) comprising the amino acid sequence set forth by SEQ ID NO:31, a VL-CDR2 comprising the amino acid sequence set forth by SEQ ID NO:32, and a VL-CDR3 comprising the amino acid sequence set forth by SEQ ID NO: 33.

8. The isolated nucleic acid of claim 1, wherein said scFv comprises a heavy chain variable region (VH) complementarity determining region (CDR) 1 (VH-CDR1) comprising the amino acid sequence set forth by SEQ ID NO:28, a VH-CDR2 comprising the amino acid sequence set forth by SEQ ID NO:29, and a VH-CDR3 comprising the amino acid sequence set forth SEQ ID NO: 30.

9. The isolated nucleic acid of claim 1, wherein said scFv comprises a light chain variable region and a heavy chain variable region oriented with said light chain variable region amino terminal to said heavy chain variable region.

10. The isolated nucleic acid of claim 1, wherein said scFv comprises a light chain variable region and a heavy chain variable region oriented with said heavy chain variable region amino terminal to said light chain variable region.

11. The isolated nucleic acid of claim 1, wherein said scFv comprises a light chain variable region and a heavy chain variable region separated by a linker.

12. The isolated nucleic acid of claim 11, wherein said linker comprises the sequence set forth by SEQ ID NO:26.

13. The isolated nucleic acid of claim 11, wherein said linker comprises the sequence set forth by SEQ ID NO:27.

14. The isolated nucleic acid of claim 1, wherein the scFv is as depicted in FIG. 1.

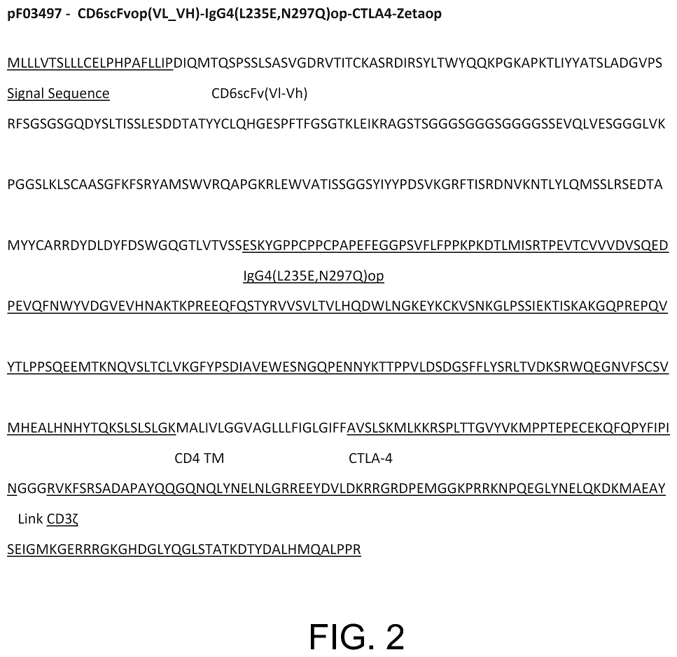

15. The isolated nucleic acid of claim 1, wherein the scFv is as depicted in FIG. 2.

16. The isolated nucleic acid of claim 14, wherein said hinge region is a human IgG Fc.

17. The isolated nucleic acid of claim 16, wherein said human IgG Fc is a human IgG4 Fc.

18. The isolated nucleic acid of claim 16, wherein said human IgG Fc is a human IgG1 Fc.

19. The isolated nucleic acid of claim 1, wherein the scFv is as depicted in FIG. 10.

20. The isolated nucleic acid of claim 1, wherein the hinge is an IgG hinge as depicted in FIG. 1.

21. The isolated nucleic acid of claim 1, wherein said scFv comprises the CDR sequences set forth by SEQ ID NOs: 20, 21, 22, 23, 24, and 25.

22. A vector comprising the nucleic acid of one of claims 1 to 21.

23. The vector of claim 22, wherein said vector is a viral vector.

24. A T lymphocyte comprising the vector of claim 22.

25. The T lymphocyte of claim 24, wherein said T lymphocyte is a regulatory T cell.

26. A chimeric antigen receptor (CAR) polypeptide comprising a single chain variable fragment (scFv) targeted to CD6, a hinge region, a transmembrane domain, a CTLA4 signaling domain and a CD3 zeta signaling domain.

27. The CAR polypeptide of claim 26, wherein said transmembrane domain comprises a CD4 transmembrane domain or a variant thereof, a CD8 transmembrane domain or a variant thereof, a CD28 transmembrane domain or a variant thereof, or a CD3ζ transmembrane domain or a variant thereof.

28. The CAR polypeptide of claim 26, wherein the svFv is as depicted in FIG. 1 or FIG. 2.

29. The CAR polypeptide of claim 28, wherein the scFv is a s depicted in FIG. 10.

30. The CAR polypeptide of claim 26, wherein said scFv comprises a light chain variable region set forth by SEQ ID NO:34.

31. The CAR polypeptide of claim 26, wherein said scFv comprises a heavy chain variable region set forth by SEQ ID NO:35.

32. The CAR polypeptide of claim 26, wherein said scFv comprises a light chain variable region (VL) complementarity determining region (CDR) 1 (VL-CDR1) comprising the amino acid sequence set forth by SEQ ID NO:31, a VL-CDR2 comprising the amino acid sequence set forth by SEQ ID NO:32, and a VL-CDR3 comprising the amino acid sequence set forth by SEQ ID NO:33.

33. The CAR polypeptide of claim 26, wherein said scFv comprises a heavy chain variable region (VH) complementarity determining region (CDR) 1 (VH-CDR1) comprising the amino acid sequence set forth by SEQ ID NO:28, a VH-CDR2 comprising the amino acid sequence set forth by SEQ ID NO:29, and a VH-CDR3 comprising the amino acid sequence set forth SEQ ID NO: 30.

34. The CAR polypeptide of claim 26, wherein said scFv is oriented with a light chain variable region at the N-terminus followed by a heavy chain variable region.

35. The CAR polypeptide of claim 26, wherein said scFv is oriented with a heavy chain variable region at the N-terminus followed by a light chain variable region.

36. The CAR polypeptide of claim 26, wherein said scFv comprises a light chain variable region and a heavy chain variable region separated by a linker.

37. The CAR polypeptide of claim 36, wherein said linker comprises the sequence set forth by SEQ ID NO:36.

38. The CAR polypeptide of claim 36, wherein said linker comprises the sequence set forth by SEQ ID NO:37.

39. The CAR polypeptide of claim 26, wherein said transmembrane domain is covalently bound to a light chain variable region of said scFv through a hinge region.

40. The CAR polypeptide of claim 26, wherein said transmembrane domain is covalently bound to a heavy chain variable region of said scFv through a hinge region.

41. The CAR polypeptide of claim 40, wherein said hinge region is a human IgG Fc.

42. The CAR polypeptide of claim 40, wherein said human IgG Fc is a human IgG4 Fc.

43. The CAR polypeptide of claim 40, wherein said human IgG Fc is a human IgG1 Fc.

44. The CAR polypeptide of claim 26, further comprising an intracellular T-cell signaling domain.

45. The CAR polypeptide of claim 44, wherein said intracellular T-cell signaling domain is a CD3ζ intracellular T-cell signaling domain.

46. The CAR polypeptide of claim 26, wherein said scFv comprises the CDR sequences set forth by SEQ ID NOs: 28, 29, 30, 31, 32, and 33.

47. The CAR polypeptide of claim 26, which is a chimeric antigen receptor (CAR).

48. A T lymphocyte comprising the CAR polypeptide of any one of claims 26 to 46.

49. The T lymphocyte of claim 48, wherein said T lymphocyte is a regulatory T cell.

50. A method of treating an autoimmune disease, said method comprising administering to a subject in need thereof an effective amount of the T lymphocyte of claim 24 or 48.

51. The method of claim 50, wherein said T lymphocyte is an autologous T lymphocyte or an allogenic T lymphocyte.

52. The method of claim 50, wherein said autoimmune disease is Type I Diabetes or Graft-versus-Host Disease.

53. The T lymphocyte of claim 49, wherein at least 70%, 80% or 90% of the cells are CD4⁺/CD25^{high}/CD127^{low/-}.

54. The T lymphocyte of claim 53, wherein at least 70%, 80% or 90% of cells are the CD6^{low/-}.


SEQUENCE LISTING (93 sequences)

SEQ ID NO: 1 - protein, 668 aa, Homo sapiens
MWLFFGITGLLTAALSGHPSPAPPDQLNTSSAESELWEPGERLPVRLTNGSSSCSGTVEVRLEASWEPACGALWDSRAAEAVCRALGCGGAEAASQLAPPTPELPPPPAAGNTSVAANATLAGAPALLCSGAEWRLCEVVEHACRSDGRRARVTCAENRALRLVDGGGACAGRVEMLEHGEWGSVCDDTWDLEDAHVVCRQLGCGWAVQALPGLHFTPGRGPIHRDQVNCSGAEAYLWDCPGLPGQHYCGHKEDAGAVCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 2 - protein, 601 aa, Homo sapiens
MWLFFGITGLLTAALSGHPSPAPPDQLNTSSAESELWEPGERLPVRLTNGSSSCSGTVEVRLEASWEPACGALWDSRAAEAVCRALGCGGAEAASQLAPPTPELPPPPAAGNTSVAANATLAGAPALLCSGAEWRLCEVVEHACRSDGRRARVTCAENRALRLVDGGGACAGRVEMLEHGEWGSVCDDTWDLEDAHVVCRQLGCGWAVQALPGLHFTPGRGPIHRDQVNCSGAEAYLWDCPGLPGQHYCGHKEDAGAVCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLIPSIVLGILLLGSLIFIAFILLRIKGKYVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSGSPSPQPDSTDNDDYDDISAA

SEQ ID NO: 3 - protein, 601 aa, Homo sapiens; identical to SEQ ID NO: 2

SEQ ID NO: 4 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 41 aa, Homo sapiens
RSKRSRGGHSDYMNMTPRRPGPTRKHYQPYAPPRDFAAYRS

SEQ ID NO: 8 - protein, 41 aa, Homo sapiens
RSKRSRLLHSDYMNMTPRRPGPTRKHYQPYAPPRDFAAYRS

SEQ ID NO: 9 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 10 - protein, 279 aa, Homo sapiens
MPTPLVHPHLPISSPRVSPFPPPAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 11 - protein, 185 aa, Homo sapiens
MGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 12 - protein, 185 aa, Homo sapiens; identical to SEQ ID NO: 11

SEQ ID NO: 13 - protein, 185 aa, Homo sapiens; identical to SEQ ID NO: 11

SEQ ID NO: 14 - protein, 42 aa, Homo sapiens
KRGRKKLLYIFKQPFMRPVQTTQEEDGCSCRFPEEEEGGCEL

SEQ ID NO: 15 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 16 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 17 - protein, 41 aa, Homo sapiens
AVSLSKMLKKRSPLTTGVYVKMPPTEPECEKQFQPYFIPIN

SEQ ID NO: 18 - DNA, 327 bp, synthetic construct
  source 1..327: mol_type = other DNA
gacatccaaatgacacaaagtcctagctctctctcagcaagtgttggcgaccgtgtgaccatcacatgtaaagcttcaagggatattcgcagctacctgacttggtaccaacaaaagcccggaaaagcgcctaaaacgcttatttactatgccaccagcctcgcagatggtgtcccctccagattttctggatcgggatcagggcaagattatagtcttacgatatcgagtcttgagtcggacgatactgccacatactactgcttacagcacggggaaagcccattcacattcggaagtggtacgaaactcgagatcaaacgggca

SEQ ID NO: 19 - DNA, 357 bp, synthetic construct
  source 1..357: mol_type = other DNA
gaagtccaattggtcgagagcggaggtgggcttgttaaaccaggaggcagtttaaaattatcatgtgctgcctcgggtttcaagttctcgcggtatgctatgtcctgggtacgccaagcacctggaaagcgtttagaatgggtggccacaattagtagtggtggttcatatatatattatcccgactccgtcaaaggaaggttcacgatttcaagggacaatgtgaagaacaccctctacttacagatgagtagtctgcgttctgaggataccgctatgtactactgtgctcggagagattacgatctggattatttcgacagctggggtcagggcacactcgttacagtatcctcg

SEQ ID NO: 20 - DNA, 60 bp, synthetic construct
  source 1..60: mol_type = other DNA
gtccaacttgttgaatcaggtggggggctggtcaaacccgggggctctctgaaactaagt

SEQ ID NO: 21 - DNA, 45 bp, synthetic construct
  source 1..45: mol_type = other DNA
ttctctcggtacgctatgtcgtgggtcagacaagcgcccggcaaa

SEQ ID NO: 22 - DNA, 57 bp, synthetic construct
  source 1..57: mol_type = other DNA
cgtgattatgatctagactactttgactcctggggtcaaggtacgctcgtgacggtt

SEQ ID NO: 23 - protein, 22 aa, Homo sapiens
MALIVLGGVAGLLLFIGLGIFF

SEQ ID NO: 24 - DNA, 66 bp, Homo sapiens
  source 1..66: mol_type = genomic DNA
atggccctgattgtgctggggggcgtcgccggcctcctgcttttcattgggctaggcatcttcttc

SEQ ID NO: 25 - protein, 21 aa, synthetic construct
IYIWAPLAGTCGVLLLSLVIT

SEQ ID NO: 26 - DNA, 54 bp, synthetic construct
  source 1..54: mol_type = other DNA
gggtcaacgtcgggcgggggttccggtggaggaagtggaggtggtggaagttct

SEQ ID NO: 27 - DNA, 60 bp, synthetic construct
  source 1..60: mol_type = other DNA
ggcggcggcggaagtggcggcggcggctcaggcggggggggttctgggggcggcggttca

SEQ ID NO: 28 - protein, 20 aa, synthetic construct
VQLVESGGGLVKPGGSLKLS

SEQ ID NO: 29 - protein, 15 aa, synthetic construct
FSRYAMSWVRQAPGK

SEQ ID NO: 30 - protein, 19 aa, synthetic construct
RDYDLDYFDSWGQGTLVTV

SEQ ID NO: 31 - protein, 38 aa, synthetic construct
CWLTKKKYSSSVHDPNGEYMFMRAVNTAKKSRLTDVTL

SEQ ID NO: 32 - protein, 42 aa, synthetic construct
ALYLLRRDQRLPPDAHKPPGGGSFRTPIQEEQADAHSTLAKI

SEQ ID NO: 33 - protein, 97 aa, synthetic construct
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 34 - protein, 109 aa, synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRA

SEQ ID NO: 35 - protein, 119 aa, synthetic construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 36 - protein, 18 aa, synthetic construct
GSTSGGGSGGGSGGGGSS

SEQ ID NO: 37 - protein, 20 aa, synthetic construct
GGGGSGGGGSGGGGSGGGGS

SEQ ID NO: 38 - protein, 246 aa, synthetic construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSGSTSGGGSGGGSGGGGSSDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRA

SEQ ID NO: 39 - protein, 248 aa, synthetic construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSGGGGSGGGGSGGGGSGGGGSDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRA

SEQ ID NO: 40 - protein, 246 aa, synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAGSTSGGGSGGGSGGGGSSEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 41 - protein, 248 aa, synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAGGGGSGGGGSGGGGSGGGGSEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 42 - DNA, 738 bp, synthetic construct
  source 1..738: mol_type = other DNA
gaagtccaattggtcgagagcggaggtgggcttgttaaaccaggaggcagtttaaaattatcatgtgctgcctcgggtttcaagttctcgcggtatgctatgtcctgggtacgccaagcacctggaaagcgtttagaatgggtggccacaattagtagtggtggttcatatatatattatcccgactccgtcaaaggaaggttcacgatttcaagggacaatgtgaagaacaccctctacttacagatgagtagtctgcgttctgaggataccgctatgtactactgtgctcggagagattacgatctggattatttcgacagctggggtcagggcacactcgttacagtatcctcggggtcaacgtcgggcgggggttccggtggaggaagtggaggtggtggaagttctgacatccaaatgacacaaagtcctagctctctctcagcaagtgttggcgaccgtgtgaccatcacatgtaaagcttcaagggatattcgcagctacctgacttggtaccaacaaaagcccggaaaagcgcctaaaacgcttatttactatgccaccagcctcgcagatggtgtcccctccagattttctggatcgggatcagggcaagattatagtcttacgatatcgagtcttgagtcggacgatactgccacatactactgcttacagcacggggaaagcccattcacattcggaagtggtacgaaactcgagatcaaacgggca

SEQ ID NO: 43 - DNA, 744 bp, synthetic construct
  source 1..744: mol_type = other DNA
gaagtccaattggtcgagagcggaggtgggcttgttaaaccaggaggcagtttaaaattatcatgtgctgcctcgggtttcaagttctcgcggtatgctatgtcctgggtacgccaagcacctggaaagcgtttagaatgggtggccacaattagtagtggtggttcatatatatattatcccgactccgtcaaaggaaggttcacgatttcaagggacaatgtgaagaacaccctctacttacagatgagtagtctgcgttctgaggataccgctatgtactactgtgctcggagagattacgatctggattatttcgacagctggggtcagggcacactcgttacagtatcctcgggcggcggcggaagtggcggcggcggctcaggcggggggggttctgggggcggcggttcagacatccaaatgacacaaagtcctagctctctctcagcaagtgttggcgaccgtgtgaccatcacatgtaaagcttcaagggatattcgcagctacctgacttggtaccaacaaaagcccggaaaagcgcctaaaacgcttatttactatgccaccagcctcgcagatggtgtcccctccagattttctggatcgggatcagggcaagattatagtcttacgatatcgagtcttgagtcggacgatactgccacatactactgcttacagcacggggaaagcccattcacattcggaagtggtacgaaactcgagatcaaacgggca

SEQ ID NO: 44 - DNA, 738 bp, synthetic construct
  source 1..738: mol_type = other DNA
gacatccaaatgacacaaagtcctagctctctctcagcaagtgttggcgaccgtgtgaccatcacatgtaaagcttcaagggatattcgcagctacctgacttggtaccaacaaaagcccggaaaagcgcctaaaacgcttatttactatgccaccagcctcgcagatggtgtcccctccagattttctggatcgggatcagggcaagattatagtcttacgatatcgagtcttgagtcggacgatactgccacatactactgcttacagcacggggaaagcccattcacattcggaagtggtacgaaactcgagatcaaacgggcagggtcaacgtcgggcgggggttccggtggaggaagtggaggtggtggaagttctgaagtccaattggtcgagagcggaggtgggcttgttaaaccaggaggcagtttaaaattatcatgtgctgcctcgggtttcaagttctcgcggtatgctatgtcctgggtacgccaagcacctggaaagcgtttagaatgggtggccacaattagtagtggtggttcatatatatattatcccgactccgtcaaaggaaggttcacgatttcaagggacaatgtgaagaacaccctctacttacagatgagtagtctgcgttctgaggataccgctatgtactactgtgctcggagagattacgatctggattatttcgacagctggggtcagggcacactcgttacagtatcctcg

SEQ ID NO: 45 - DNA, 744 bp, synthetic construct
  source 1..744: mol_type = other DNA
gacatccaaatgacacaaagtcctagctctctctcagcaagtgttggcgaccgtgtgaccatcacatgtaaagcttcaagggatattcgcagctacctgacttggtaccaacaaaagcccggaaaagcgcctaaaacgcttatttactatgccaccagcctcgcagatggtgtcccctccagattttctggatcgggatcagggcaagattatagtcttacgatatcgagtcttgagtcggacgatactgccacatactactgcttacagcacggggaaagcccattcacattcggaagtggtacgaaactcgagatcaaacgggcaggcggcggcggaagtggcggcggcggctcaggcggggggggttctgggggcggcggttcagaagtccaattggtcgagagcggaggtgggcttgttaaaccaggaggcagtttaaaattatcatgtgctgcctcgggtttcaagttctcgcggtatgctatgtcctgggtacgccaagcacctggaaagcgtttagaatgggtggccacaattagtagtggtggttcatatatatattatcccgactccgtcaaaggaaggttcacgatttcaagggacaatgtgaagaacaccctctacttacagatgagtagtctgcgttctgaggataccgctatgtactactgtgctcggagagattacgatctggattatttcgacagctggggtcagggcacactcgttacagtatcctcg

SEQ ID NO: 46 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 47 - protein, 198 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAGNRRRVCKCPRPVVKSGDKPSLSARYV

SEQ ID NO: 48 - protein, 235 aa, Homo sapiens; identical to SEQ ID NO: 46

SEQ ID NO: 49 - protein, 246 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQMSREYGFGVLLQKDIGQ

SEQ ID NO: 50 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 51 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 52 - protein, 213 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGRRRRARLRFMKQPQGEGISGTFVPQCLHGYYSNTTTSQKLLNPWILKT

SEQ ID NO: 53 - protein, 221 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 54 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 55 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 57 - protein, 123 aa, Homo sapiens; identical to SEQ ID NO: 4

SEQ ID NO: 58 - protein, 101 aa, Homo sapiens; identical to SEQ ID NO: 5

SEQ ID NO: 59 - protein, 220 aa, Homo sapiens; identical to SEQ ID NO: 6

SEQ ID NO: 60 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 61 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 62 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 63 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 64 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 65 - protein, 255 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCCWRCRRRPKTPEAASSPRKSGASDRQRRRGGWETCGCEPGRPPGPPTAASPSPGAPQAAGALRSALGRALLPWQQKWVQEGGSDQRPGPCSSAAAAGPCRRERETQSWPPSSLAGPDGVGS

SEQ ID NO: 66 - protein, 234 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRKTQLLLEVPPSTEDARSCQFPEEERGERSAEEKGRLGDLWV

SEQ ID NO: 67 - protein, 57 aa, Homo sapiens
QLGLHIWQLRSQCMWPRETQLLLEVPPSTEDARSCQFPEEERGERSAEEKGRLGDLW

SEQ ID NO: 68 - protein, 118 aa, synthetic construct
EVQLVESGGGLVKPGGSLKLSCAASGFKFSRYAMSWVRQTPEKRLEWVATISSGGSYIYYPDSVKGRFTISRDNVKNTLYLQMSSLRSEDTAMYYCARRDYDLDYFDSWGQGTTLTVS

SEQ ID NO: 69 - protein, 109 aa, synthetic construct
DIKMTQSPSSMYASLGERVTITCKASRDIRSYLTWYQQKPWKSPKTLIYYATSLADGVPSRFSGSGSGQDYSLTISSLESDDTATYYCLQHGESPFTFGSGTKLEIKRA

SEQ ID NO: 70 - protein, 22 aa, synthetic construct
MLLLVTSLLLCELPHPAFLLIP

SEQ ID NO: 71 - protein, 24 aa, Homo sapiens
LEGGGEGRGSLLTCGDVEENPGPR

SEQ ID NO: 72 - skipped

SEQ ID NO: 73 - protein, 323 aa, Homo sapiens
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 74 - skipped

SEQ ID NO: 75 - protein, 68 aa, Homo sapiens
IQMTQSPSSLSASVGDRVTITCKASRDIRSYLTWYQQKPGKAPKTLIYYATSLADGVPSRFSGSGSGQ

SEQ ID NO: 76 - protein, 32 aa, synthetic construct
DIQMTQSPSSLSASVGDRVTITCKASRDIRSY

SEQ ID NO: 77 - protein, 65 aa, synthetic construct
LTWYQQKPGKAPKTLIYYATSLADGVPSRFSGSGSGQDYSLTISSLESDDTATYYCLQHGESPFT

SEQ ID NO: 78 - protein, 12 aa, synthetic construct
FGSGTKLEIKRA

SEQ ID NO: 79 - protein, 35 aa, synthetic construct
EVQLVESGGGLVKPGGSLKLSCAASGFKFSRYAMS

SEQ ID NO: 80 - protein, 20 aa, synthetic construct
WVRQAPGKRLEWVATISSGG

SEQ ID NO: 81 - protein, 53 aa, synthetic construct
SYIYYPDSVKGRFTISRDNVKNTLYLQMSSLRSEDTAMYYCARRDYDLDYFDS

SEQ ID NO: 82 - protein, 11 aa, synthetic construct
WGQGTLVTVSS

SEQ ID NO: 83 - protein, 653 aa, synthetic construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSGSTSGGGSGGGSGGGGSSDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFAVSLSKMLKKRSPLTTGVYVKMPPTEPECEKQFQPYFIPINGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 675 aa, synthetic construct
MLLLVTSLLLCELPHPAFLLIPEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSGSTSGGGSGGGSGGGGSSDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFAVSLSKMLKKRSPLTTGVYVKMPPTEPECEKQFQPYFIPINGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 653 aa, synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAGSTSGGGSGGGSGGGGSSEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFAVSLSKMLKKRSPLTTGVYVKMPPTEPECEKQFQPYFIPINGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 675 aa, synthetic construct
MLLLVTSLLLCELPHPAFLLIPDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAGSTSGGGSGGGSGGGGSSEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFAVSLSKMLKKRSPLTTGVYVKMPPTEPECEKQFQPYFIPINGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 654 aa, synthetic construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSGSTSGGGSGGGSGGGGSSDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 676 aa, synthetic construct
MLLLVTSLLLCELPHPAFLLIPEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSGSTSGGGSGGGSGGGGSSDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 654 aa, synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAGSTSGGGSGGGSGGGGSSEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 676 aa, synthetic construct
MLLLVTSLLLCELPHPAFLLIPDIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRAGSTSGGGSGGGSGGGGSSEVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 229 aa, Homo sapiens
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 68 aa, Homo sapiens; identical to SEQ ID NO: 75